(19) United States
(12) Patent Application Publication
(10) Pub. No.: US 2002/0001805 A1
(43) Pub. Date: Jan. 3, 2002

(54) Immunogenic ovarian cancer genes

(76) Inventors: Richard Bruce Roden, Washington, DC (US); Honami Naora, Baltimore, MD (US)

Correspondence Address: BROBECK, PHLEGER & HARRISON, LLP, ATTN: INTELLECTUAL PROPERTY DEPARTMENT, 1333 H STREET, N.W. SUITE 800, WASHINGTON, DC, 20005, US

(21) Appl. No.: 09/805,177

(22) Filed: Mar. 14, 2001

Related U.S. Application Data
(60) Provisional application No. 60/189,226, filed on Mar. 14, 2000.
(60) Provisional application No. 60/258,452, filed on Dec. 28, 2000.

(51) Int. Cl. C12Q 1/68; G01N 33/574; C07H 21/04

(52) U.S. Cl. 435/6; 435/7.23; 435/69.1; 435/325; 530/350; 536/23.5

(57) ABSTRACT

The present invention is based on the discovery of autoantibodies in cancer patients specific for a number of antigens that are normally intracellular, including homeobox protein HOXA7, homeobox protein HOXB7, ADP-ribosylation factor 1 (Arf-1), ATP-dependent iron transporter ABC-7, and a novel protein encoded by a EcoRI/XhoI fragment of bacteriophage λ clone 44B.1 deposited under ATCC accession No. [N]. The presence of these autoantibodies can be correlated with neoplastic processes in patients, and therefore detection of autoantibodies (or detection of expression of the antigens by other means) can be used as a component of a cancer screening program. The present invention provides such screening assays. In addition, the studies leading to the identification of the predictive autoantigens have also succeeded in identifying a hitherto unknown antigen that is disclosed herein.

This application is based on U.S. Provisional Application No. 60/189,226, filed Mar. 14, 2000 and U.S. Provisional Application No. 60/258,452, filed Dec. 28, 2000, which are each incorporated herein by reference in their entirety.

### BACKGROUND OF THE INVENTION

1. Field of the Invention

This invention is directed to the field of cancer diagnostics and therapeutics. In particular, this invention provides novel cancer related antigens and novel relations between known antigens and cancer for purposes of diagnosis and therapy.

2. Related Art

Cancers arise through accumulation of a series of genetic and epigenetic changes that disrupt normal control of cell growth (Auersperg, N., Edelson, M. I., Mok, S. C., Johnson, S. W., and Hamilton, T. C., “The Biology of Ovarian Cancer,” Semin. Oncol., 25:281-304, 1998). These molecular alterations result in changes in the level of gene expression and in the structure, function and activity of gene products. Such alterations affect many cellular processes, including the quantity and nature of molecules that are released from the cell. Detection of such molecules in the peripheral circulation has proved valuable for the diagnosis and prognosis of various cancers, a good example being plasma PSA tests for prostate cancer (Woolf, S. H., “Screening for Prostate Cancer with Prostate-Specific Antigen. An Examination of the Evidence,” N. Engl. J. Med., 333:1401-1405, 1995). To date, none of the various molecules that have been detected at elevated levels in the sera of ovarian cancer patients have alone been established as a sufficiently consistent and specific early detection biomarker. Elevated levels of CA125, regarded as the most specific serum marker for ovarian cancer, occurs only in 50% of clinically detected Stage I ovarian cancer patients, and can occur in healthy women, and women with benign cysts and other unrelated clinical conditions (Jacobs, I. and Bast, R. C., Jr., “The CA 125 Tumour-Associated Antigen: A Review of the Literature,” Hum. Repro., 4:332-337, 1989). Therefore, identifying novel serum biomarkers of ovarian cancer is critical and particularly challenging.

Antibody responses against tumor molecules: The human immune system is able to recognize minute quantities of foreign antigen and amplify this foreign signal by generating antibodies (Old, L. J. and Chen, Y. T., “New Paths in Human Cancer Serology,” . Exp. Med., 187:1163-1167, 1998). Patients with various types of cancer have been found to generate antibodies against specific proteins expressed by their tumors (Sahin, u., Tureci, O., Schmitt, H., Cochlovius, B., Johannes, T., Schmits, R., Stenner, F., Luo, G., Schobert, I., and Pfreundschuh, M., “Human Neoplasms Elicit Multiple Specific Immune Responses in the Autologous Host,” Proc. Natl. Acad. Sci. USA, 92:11810-11813, 1995). Furthermore, such antibodies are generally absent in sera of healthy individuals (Stockert, E., Jager, E., Chen, Y. T., Scanlan, M. J., Gout, I., Karbach, J., Arand, M., Knuth, A., and Old, L. J., “A Survey of the Humoral Immune Response of Cancer Patients to a Panel of Human Tumor Antigens,” J. Exp. Med., 187:1349-1354, 1998). Sera from patients have been used to screen cDNA expression libraries constructed from their tumor to identify tumor antigens. This approach, termed SEREX (serological identification of antigens by recombinant expression cloning), has uncovered a wealth of antigens associated with a variety of tumors e.g. of melanoma, breast, kidney, testis, colon (Brass, N., Heckel, D., Sahin, U., Pfreundschuh, M., Sybrecht, G. W., and Meese, E., “Translation Initiation Factor eIF-4gamma is Encoded by an Amplified Gene and Induces an Immune Response in Squamous Cell Lung Carcinoma,” Hum. Mol. Genet., 6:33-39, 1997; Old and Chen, 1998; Scanlan. M. J., Chen, Y. T., Williamson, B., Gure, A. O., Stockert, E., Gordan, J. D., Tureci, O., Sahin, U., Pfreundschuh, M., and Old, L. J., “Characterization of Human Colon Cancer Antigens Recognized by Autologous Antibodies,” Int. J. Cancer, 76:652.658, 1998).

Several studies demonstrate that ovarian cancer patients can generate tumor-reactive antibody (Taylor, D. D. and Gercel-Taylor, C., “Tumor-Reactive Immunoglobulins in Ovarian Cancer: Diagnostic and Therapeutic Significance?” Oncol. Rep., 5:1519-1524, 1998). Antibody reactive with tumor but not normal ovary can be isolated from membrane fractions in ascites (Daunter, B., Jaa-Kwee, D., Miklosi, S., and Writht, G., “Ovarian Tumor Antigens: Preliminary Histological Investigation,” Gynecol. Oncol., 23:364-370, 1986; Taylor, D. D., Homesley, H. D., and Doellgast, G. J., “Identification of Antigenic Components Recognized by ‘Membrane-Bound’ Antibodies from Ovarian Cancer Patients,” Am. J. Reprod. Immunol., 6:179-184, 1984). Dawson et al identified multiple ovarian tumor antigens in immune complexes with patient antibody (Dawson, J. R., Lutz, P. M., and Shau, H., “The Humoral Response to Gynecologic Malignancies and Its Role in the Regulation of Tumor Growth: A Review,” Am. J. Reprod. Immunol., 3:12-17, 1983). Ovarian cancer patients generate autoantibody to p53 (Angelopoulou, K., Rosen, B., Stratis, M., Yu, H., Solomou, M., and Diamandis, E. P., “Circulating Antibodies Against p53 Protein in Patients with Ovarian Carcinoma. Correlation with Clinicopathologic Features and Survival., Cancer, 78:2146-2152, 1996; Gadducci, A., Ferdeghini, M., Buttitta, F., Cosio, S., Fanucchi, Al, Annicchiarico, C., and Genazzani, A. R., “Serum Anti-p53 Antibodies in the Follow-Up of Patients with Advanced Ovarian Carcinoma,” Anticancer Res., 18:3763-3765, 1998; Gadducci, A., Ferdeghini, M., Buttitta, F., Fanucchi, A., Annicchiarico, C., Prontera, C., Bevilacqua, G., and Genazzani, A. R., “Preoperative Serum Antibodies Against the p53 Protein in Patients with Ovarian and Endometrial Cancer,” Anticancer Res., 16:3519-3523, 1996). Alloantibody to NY-ESO-1, an antigen identified in esophageal cancer, was detected in 4 of 32 ovarian cancer patients and none of 70 controls (Stockert et al., 1998). Other tumor antigens that are common among ovarian cancer patients have been identified; of 25 sera derived from ovarian cancer patients, 12 (48%) recognized cathepsin D and 10 (40%) reacted with glucose-regulated protein 78 (GRP78) (Chinni, S. R., Falchetto, R., Gercel-Taylor, C. Shabanowitz, J., Hunt, D. F., and Taylor, D. D., “Humoral Immune Responses to Cathepsin D and Glucose-Regulated Protein 78 in Ovarian Cancer Patients,” Clin. Cancer Res., 3:1557-1564, 1997). Neither ovarian cancer antigen was recognized by antibody in sera of normal controls. Furthermore, it appears that both of these antigens contain tumor-specific epitopes since neither cathepsin D nor GRP78 derived from normal tissue were recognized by sera from ovarian cancer patients (Chinni et al., 1997). Detection of these autologous antibody responses to ovarian cancer antigens appears to have prognostic significance (Chinni, S. R., Gercel-Taylor, C., Conner, G. E., and Taylor, D. D., “Cathepsin D Antigenic Epitopes Identified by the Humoral Responses of Ovarian Cancer Patients,” Cancer Immunol. Immunother, 46:48-54, 1998). However, the clinical significance of detection of autologous anti-tumor antibodies requires further analysis and must be assessed for each antigen.

### SUMMARY OF THE INVENTION

The present inventors have discovered autoantibodies in cancer patients specific for a number of antigens that are normally intracellular. The presence of these autoantibodies can be correlated with neoplastic processes in patients, and therefore detection of autoantibodies (or detection of expression of the antigens by other means) can be used as a component of a cancer screening program. The present invention provides such screening assays. In addition, the studies leading to the identification of the predictive autoantigens have also succeeded in identifying a hitherto unknown antigen that is disclosed herein.

In one embodiment, this invention provides a substantially pure nucleic acid which is homologous with a EcoRI/XhoI fragment isolated from λ phage clone 44B.1 deposited under ATCC accession No. [N] . Sequences of this invention are homologous as determined by a sequence alignment score of greater than 200 as calculated by BLASTN 2.1.2. Alternatively, this invention provides a substantially pure nucleic acid which hybridizes to a EcoRI/XhoI fragment isolated from λ phage clone 44B.1 deposited under ATCC accession No. [N] under stringent conditions, wherein stringent conditions comprise 0.5 M NaHPO₄/1 mM EDTA/7% (w/v) SDS at 55° C., preferably 60° C., more preferably 65° C. The nucleic acid may be DNA or RNA, and may be produced by recombinant methods. The nucleic acid is preferably at least 15 nucleotides in length, and may encode the entire amino acid sequence encoded by the EcoRI/XhoI fragment of clone 44B.1. The invention also includes a pair of nucleic acid primers comprising at least 10 contiguous nucleotides selected from or complementary to portion of a EcoRI/XhoI fragment isolated from bacteriophage clone 44B.1 deposited under ATCC accession No. [N]. The primers of this invention will produce an amplified nucleic acid comprising at least 18 contiguous nucleotides of the EcoRI/XhoI fragment isolated from bacteriophage clone 44B.1 deposited under ATCC accession No. [N]. The invention also provides replicons (e.g., nucleic acid vectors) comprising a sequence of at least 18 contiguous nucleotides selected from the sequence of a EcoRI/XhoI fragment isolated from bacteriophage clone 44B. 1 deposited under ATCC accession No. [N] or its complement under control of a promoter, as well as recombinant cells containing the replicon.

In another embodiment, this invention provides a substantially pure polypeptide comprising an amino acid sequence encoded by a EcoRI/XhoI fragment isolated from bacteriophage clone 44B. 1 deposited under ATCC accession No. [N]; preferably, the polypeptide comprises at least one epitope, typically containing at least 9 amino acids. In a particular embodiment, the peptide of this invention consists essentially of an amino sequence encoded by said EcoRI/XhoI fragment.

In yet another embodiment, this invention provides an antibody which specifically binds to a mammalian protein comprising an amino acid sequence encoded by a EcoRI/XhoI fragment isolated from clone 44B.1 deposited under ATCC accession No. [N ]. The antibody may be an isolated polyclonal antiserum, a preparation of purified polyclonal antibodies, or a preparation containing one or more monoclonal antibodies.

In still another embodiment, this invention provides a method for selecting variant nucleic acid sequences comprising (a) screening mammalian DNA or RNA with a nucleic acid probe comprising the nucleic acid of claim 1 or claim 2, (b) sequencing the DNA or RNA obtained in said screening, and (c) selecting DNA or RNA having sequences that differ from the nucleic acid sequence in a EcoRI/XhoI fragment of bacteriophage clone 44B.1 deposited under ATCC accession No. [N] by at least one nucleotide.

In yet another embodiment, this invention provides a method of screening for cancer in an individual comprising determining whether cells in the individual are expressing a gene product encoded by a EcoRI/XhoI fragment isolated from clone 44B.1 deposited under ATCC accession No. [N], expression of this product being correlated with an increased likelihood of cancer in the individual.

In still another embodiment, this invention provides a nucleic acid encoding HOXB7, having the nucleic acid sequence in FIG. 12, and the protein encoded by the nucleic acid sequence, as well as equivalents, i.e., variants of both the nucleic acid and the protein so long as the variants differ by at least one residue (nucleotide or amino acid, respectively) from other known HOXB7 gene products (e.g., Genbank sequences AF287967, AF284825, NM004502, and U15407). This invention also provides a method of screening for cancer in an individual comprising determining whether cells in the individual are expressing a product encoded by the nucleic acid of FIG. 12, expression of this product being correlated with an increased likelihood of cancer in the individual.

In yet another embodiment, this invention provides a method of screening for cancer, other than breast cancer or melanoma, in an individual comprising determining whether cells in the individual are expressing a gene product of HOXB7 gene, expression of this gene product being correlated with an increased likelihood of cancer in the individual.

In still another embodiment, this invention provides a method of screening for neoplasms, including cancer, other than renal cell carcinoma or colon cancer, especially ovarian neoplasms, in an individual comprising determining whether cells in the individual are expressing a gene product of HOXA7 gene, expression of this gene product being correlated with an increased likelihood of neoplasm, including ovarian cancer or benign serous cystadenoma, in the individual. Alternatively, this invention provides screening methods for cancer in an individual comprising determining whether cells in the individual are expressing a product consisting of ATP-dependent iron transporter ABC-7 (Genbank ID AF133659) or a product consisting of ADP-ribosylation factor 1 (Arf-1) (Genbank ID AF052179), expression of either of these products being correlated with an increased likelihood of cancer in the individual.

In yet another embodiment, this invention provides a method of screening for cancer in an individual comprising determining whether cells in the individual are simultaneously expressing two or more gene products selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage clone 44B.1 deposited under ATCC accession No. [N], expression of a plurality of these gene products being correlated with an increased likelihood of cancer in the individual.

In a preferred mode, the screening methods of this invention comprise (a) providing a histologic section of tissue from the individual; (b) contacting said histologic section with antibody which specifically binds said product; and (c) determining said antibody specifically binds to the histologic section, whereby specific binding of said antibody to the histologic section correlates with increased likelihood of cancer in the individual. In another preferred mode, the screening methods of this invention comprise (a) providing a sample of tissue from the individual; and (b) determining, in said sample, level of expression of a gene product having the sequence of said product, whereby expression of said product in the sample correlates with increased likelihood of cancer in the individual. In one preferred mode, the gene product is mRNA, and the mRNA may be extracted from said sample and quantitated, or the level of MnRNA may be determined by in situ hybridization to a section of the tissue sample. Alternatively, the mRNA may be quantitated by reverse transcriptase-polymerase chain reaction. Preferably, the tissue is a cancer, which may be ovarian cancer, breast cancer or melanoma.

In still another embodiment, this invention provides a kit for screening human samples according to the method of this invention, where the kit comprises one or more containers that hold an antibody which specifically binds to one or more epitopes found on said product; and a reagent means for detecting the antibody. Alternatively, the kit may comprise one or more containers holding a nucleotide probe which hybridizes to mRNA encoding said product; and reagent means for detecting the nucleotide probe.

In particularly preferred embodiment, this invention provides a method of screening for cancer in an individual comprising obtaining a sample of bodily fluid from the individual and determining whether or not the sample contains antibodies specific for one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRlIXhol fragment of bacteriophage clone 44B.1 deposited under ATCC accession No. [N], the presence of antibodies to any one of these proteins being correlated with an increased likelihood of cancer in the individual. Alternatively, the invention provides a method of screening for cancer in an individual comprising obtaining a sample of bodily fluid from the individual and determining whether or not the sample contains one or more proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage clone 44B.1 deposited under ATCC accession No. [N], the presence of any one of these proteins in the bodily fluid being correlated with an increased likelihood of cancer in the individual.

In yet another embodiment, this invention provides a method of cancer therapy comprising immunizing an individual with an immunogenic composition to elicit an immune response to one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage clone 44B.1 deposited under ATCC accession No. [N]. Preferably, the immunogenic composition comprises at least one epitope of one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage clone 44B.1 deposited under ATCC accession No. [N]. In a preferred mode, the immune response is a cellular immune response.

### BRIEF DESCRIPTION OF THE FIGURES

FIG. 1. Detection of tumor antigens by Western blot analysis using patient sera.

FIG. 2. Size selection and gel purification of OV1063 cDNA-linker.

FIG. 3. Recombinants from a single clone transferred to an IPTG-soaked membrane and incubated with patient antisera.

FIG. 4. Analysis of HOXA7 and B7 mRNA transcript in ovarian surface epithelium and tumors.

FIG. 5. Expression of HOXA7 in normal ovary and ovarian tumors.

FIG. 6. A. Phage blot of HOXA7 bacteriophage plaques using sera from patients and controls.

FIG. 6. B. Western blot of recombinant HOXA7 and HOXB7 using patient serum.

FIG. 7. ELISA of antibodies to HOXB7 or control antigen in sera of ovarian cancer patients and controls.

FIG. 8. ELISA of antibodies to HOXA7 or control antigen in sera of ovarian tumor patients and controls.

FIG. 9. HOXB7 expression levels (Panel A) and morphology (Panels B & C) of transfected IOSE-29 cells.

FIG. 10. Growth characteristics of transfected cells.

FIG. 11. Increased bFGF production by HOXB7-transfected cells.

FIG. 12. Sequence of a HOXB7 Variant. (SEQ ID NO: 1).

### DETAILED DESCRIPTION OF THE EMBODIMENTS

Definitions

In describing the present invention, the following terminology is used in accordance with the definitions set out below.

“Nucleic acid” will refer to either or both DNA and RNA, unless the context makes clear that only one form is intended.

A “replicon” is any genetic element (e.g., plasmid, chromosome, virus) that functions as an autonomous unit of nucleic acid replication in vivo; i.e., capable of replication under its own control.

Vectors are used to introduce a foreign substance, such as DNA, RNA or protein, into an organism. Typical vectors include recombinant viruses (for nucleic acids) and VP22 from herpes simplex virus 1 (for protein). A “DNA vector” is a replicon, such as plasmid, phage or cosmid, to which another DNA segment may be attached so as to bring about the replication of the attached segment.

An “expression vector” is a nucleic acid vector which contains regulatory sequences which will direct protein synthesis by an appropriate host cell. This usually means a promoter to bind RNA polyrnerase and initiate transcription of MRNA, as well as ribosome binding sites and initiation signals to direct translation of the niRNA into a polypeptide. Incorporation of a nucleic acid sequence into an expression vector at the proper site and in correct reading frame, followed by transfection of an appropriate host cell by the vector, enables the production of a protein encoded by the sequence.

An expression vector may alternatively contain an antisense sequence, where a small DNA fragment, corresponding to all or part of an mRNA sequence, is inserted in opposite orientation into the vector after a promoter. As a result, the inserted DNA will be transcribed to produce an RNA which is complementary to and capable of binding or hybridizing to the mRNA. Upon binding to the mRNA, translation of the mRNA is prevented, and consequently the protein coded for by the MRNA is not produced. Production and use of antisense expression vectors is described in more detail in U.S. Pat. No. 5,107,065 and U.S. Pat. No. 5,190,931, both of which are incorporated herein by reference.

A “gene product” as contemplated herein includes both mRNA and the polypeptide expressed from it.

The degree of similarity between two nucleic acids may be described as their degree of homology (based on comparison of the chemical structure of the nucleic acids, as expressed by the sequence of nucleotides making up the nucleic acid) or functionally (based on whether two nucleic acids will hybridize to form a double-stranded complex). Similarity described functionally includes information on the conditions under which hybridization occurs, for example, two sequences are said to hybidize under stringent conditions when two single strands will hybridize when incubated in 0.5 M NaHPO₄/1mM EDTA/7% (w/v) SDS at 55° C. or above. Hybridization under highly stringent conditions involves incubation in the same medium at temperatures of 60° C. and above, and very high stringency at temperatures of 65° C. and above. Homology is defined by the sequence alignment score of two sequences as calculated by the BLASTN 2.1.2 program (National Library of Medicine, Nov. 13, 2000). High homology is represented by a score greater than 200.

The term “specific binding” as used herein refers to one molecule binding another molecule with high specificity, as for example an antigen and an antibody elicited by immunization with the antigen. In general, the specific binding partners must bind with sufficient affinity to immobilize the analyte (e.g., copy/complementary strand duplex, in the case of capture probes) under the hybridization conditions. Specific binding pairs are known in the art, and include, for example, biotin and avidin or streptavidin, IgG and protein A, the numerous known receptor-ligand couples, and complementary polynucleotide strands. In the case of complementary polynucleotide binding partners, the partners are normally at least about 15 bases in length, and may be at least 40 bases in length; in addition, they have a content of Gs and Cs of at least about 40% and as much as about 60%. The polynucleotides may be composed of DNA, RNA, or synthetic nucleotide analogs.

A macromolecular component is “substantially pure” if the level of similar macromolecules is no greater than 20% that of the substantially pure component. In other words, a substantially pure nucleic acid species makes up at least 80% of the nucleic acids in the composition.

An “epitope” is a structure, usually made up of a short peptide sequence or oligosaccharide, that is specifically recognized or specifically bound by a component of the immune system. T-cell epitopes have generally been shown to be linear oligopeptides, typically having about nine amino acids. Two epitopes correspond to each other if they can be specifically bound by the same antibody. Two antibodies correspond to each other if both are capable of binding to the same epitope, and binding of one antibody to its epitope prevents binding by the other antibody. Antibodies as contemplated by this invention include intact immunoglobulin molecules, immunoglobulin fragments that retain specific binding capacity, humanized antibodies, single chain antibodies, and compositions containing either polyclonal or monoclonal antibodies.

As used herein, a “biological sample” refers to a sample of tissue or fluid isolated from a individual, including but not limited to, for example, plasma, serum, spinal fluid, lymph fluid, the external sections of the skin, respiratory, intestinal, and genitourinary tracts, tears, saliva, milk, blood cells, tumors, organs, and also samples of in vivo cell culture constituents (including but not limited to conditioned medium resulting from the growth of cells in cell culture medium, putatively virally infected cells, recombinant cells, and cell components).

“Human tissue” is an aggregate of human cells which may constitute a solid mass. This term also encompasses a suspension of human cells, such as blood cells, or a human cell line. Human biological fluid includes plasma, serum, spinal fluid, lymph fluid, the external sections of the skin, respiratory, intestinal, and genitourinary tracts, tears, saliva, and milk, but not blood cells or other cells.

A “tumor” as discussed herein is a new growth of tissue in which the multiplication of cells is uncontrolled and progressive. “Neoplasm” refers to any new and abnormal growth of tissue which is uncontrolled and progressive; neoplastic growth includes both fatal and non-fatal tumors. “Cancer” is a cellular tumor, the natural course of which is fatal.

The practice of the present invention employs, unless otherwise indicated, conventional molecular biology, microbiology, and recombinant DNA techniques within the skill of the art. Such techniques are well known to the skilled worker and are explained fully in the literature. See, e.g., Sambrook, et al., “Molecular Cloning: a Laboratory Manual” Cold Spring Harbor Press (1989) and “Current Protocols In Molecular Biology,” Ausubel, et al., eds., Current Protocols, 1994.

Novel Cancer-Related Antigen

The inventors have identified an antigen which has not been reported in normal (non-malignant) cells, but has now been found in tumor cell lines and reacts with antibodies in sera from cancer patients. A clone containing the nucleic acid sequence encoding this antigen has been deposited (see Example 4), and the skilled worker may obtain samples of this antigen by transferring the EcoRI/XhoI fragment from the deposited clone to an expression vector using standard recombinant DNA methodology (see, e.g., Sambrook, et al.) and expressing the polypeptide in a conventional host expression system. Alternatively, the relevant fragment (described in Examples 2-4) may be used to probe a human cDNA library, and members of the library which hybridize with the fragment may be used as a source of nucleic acid encoding the novel antigen. Cloning the sequence, inserting it into an expression vector, and introducing the expression vector into a suitable host for expression may be accomplished by the skilled person using routine procedures. Samples of the process for expressing polypeptides from cloned nucleic acid sequences are provided in the Examples, and alternative procedures to produce intermediate vectors and expression systems are within the skill of the art. Cloning vectors containing sequence encoding the antigen, host cells containing the sequence, and antigenic polypeptides expressed by the cells according to these routine procedures are within the contemplation of this invention.

Antibodies that specifically bind the novel antigen of this invention may be prepared according to well known procedures in the field of immunology (see, e.g., Davis, et al., eds., Microbiology, 3rd ed., Hagerstown, Harper & Row, 1980, and Paul, ed., Fundamental Immunology, 3rd ed., New York, Raven Press, 1993). Suitable animals may be immunized with antigen produced by a recombinant host cell (e.g., mice, rabbits, sheep, goats, turkeys, chickens, or primates, including humans). The antigen (immunogen) may be introduced into the animal in saline buffer or accompanied by one or more adjuvants, or an expression vector may be introduced into the animal for production of the antigen in vivo. Antibodies may be prepared and purified according to the processes described in U.S. Pat. Nos. 5,734,002, and 5,759,791, incorporated herein by reference, by substituting the antigens of this invention into the described processes.

Macromolecular Variants

“Variants” of a biological macromolecule disclosed herein may be prepared by the skilled worker using routine techniques. For example, the skilled worker will be able to prepare fragmentary nucleic acids and polypeptides having sequences of nucleotides and amino acids, respectively, that correspond to portions of the novel sequence disclosed herein (“fragmentary variants”). These fragmentary variants may be prepared by chemical synthesis, by cleavage from larger macromolecules, or by recombinant techniques. The sequence of these fragments may vary from the exact sequence deposited, so long as they hybridize with the deposited sequence under stringent, or highly stringent, conditions (“stringency variants”) and/or meet the homology condition (sequence alignment score of at least 200 as calculated by BLASTN 2.1.2 (“homology variants”)). In particular, the present invention contemplates fragments which include at least one epitope bound by antibodies or by MHC complexes (“homo-epitopic variants”).

Examples of proteins that can be used in the present invention include polypeptides with minor amino acid variations from the natural amino acid sequence of the protein (“substitution variants”); in particular, conservative amino acid replacements are contemplated. Conservative replacements are those that take place within a family of amino acids that are related in their side chains. Genetically encoded amino acids are generally divided into four families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cystine, serine, threonine, tyrosine. Phenylalanine, tryptophan, and tyrosine are sometimes classified jointly as aromatic amino acids. For example, it is reasonably predictable that an isolated replacement of a leucine with an isoleucine or valine, an aspartate with a glutamate, a threonine with a serine, or a similar conservative replacement of an amino acid with a structurally related amino acid will not have a major effect on the biological activity. Polypeptide molecules having substantially the same amino acid sequence as the protein but possessing minor amino acid substitutions that do not substantially affect the functional aspects are within the definition of the protein.

Selection of fragments containing at least one epitope (i.e., homo-epitopic variants) may be accomplished on a theoretical basis or based on empiric functional determinations or a combination of the two approaches, e.g., as described in Westhof, et al. (“Correlation Between Segmental Mobility and the Location of Antigenic Determinants in Proteins,” Nature, 311:123-126, 1984), incorporated herein by reference. Empiric selections typically begin with production of a collection of polypeptide fragments of polypeptide sequences according to this invention, which are tested to determine whether any fragments in the collection contain epitopes or not. Testing such fragments for the presence of epitopes is within the skill of the art in the field of immunology, in view of the guidance provided herein. Suitable tests include incubation with polyclonal antisera elicited by immunization using the novel protein and detection of complexes between one or more fragments and antibodies in the serum. Similar studies to detect epitopes may be performed by monitoring T cell activating activity to determine which fragments in a collection demonstrate the activity.

Preferably, theoretical identification of epitopic fragments will be confirmed empirically. Nucleic acid fragments which contain epitopes may be selected based on sequences that encode polypeptide fragments having epitopes as determined by testing the polypeptide. Correlation between nucleic acid and polypeptide sequences may be based on comparison of the sequences through the genetic code, or by production of the polypeptide from the nucleic acids using recombinant techniques.

Variants of the macromolecules described for this invention may include any one, or a plurality, of the deviations from the sequence of the macromolecule described above.

Diagnostic Assays

The novel antigen was found in association with tumor cells, and detection of its expression in an individual may be taken to suggest that the individual harbors tumor cells. Thus, this novel antigen may form the basis for screening assays in which determination that the novel antigen is expressed by an individual provides supporting data for a diagnosis of cancer in the individual, or for prognostic monitoring of progress in an individual after diagnosis.

Detecting expression of the novel antigen of this invention or its variants may be accomplished at the DNA, RNA, or protein level. Alternatively, expression of the novel antigen may be deduced from presence of autoimmune antibodies specific for the antigen. Antibodies can be prepared by immunizing mammals with peptides expressed from nucleic acid sequences corresponding to the novel antigen, as indicated above, and selecting those antibodies specific to epitopes found on the novel antigen using techniques that are well known to those skilled in the art. These antibodies can detect expression of the novel antigen by a variety of immunoassay techniques. The nucleotide probe sequences provided by the invention can be used to detect expression of mRNA corresponding to the novel antigen in accordance with any of the standard techniques. Expression may be detected either by in situ hybridization or by extraction and detection of MRNA. Autoimmune antibodies can be detected using epitope-containing peptides of the novel antigen to bind antibodies in biological fluid from a patient in any suitable immunoassay format. The particular procedures for gene probe assays and immunoassays will be well-known to those skilled in the art.

Immunoassays

A protein cross-reactive with the novel antigen can be quantitated in a biological fluid, such as serum, plasma, effusions, ascites, urine, cerebrospinal fluid, semen, breast aspirates and fluids of ovarian origin, using any protein detection means known in the art. Preferred methods employ immunological detection means. These include: radioimmunoassay, enzyme linked immuno-sorbent assay (ELISA), complement fixation, nephelometric assay, immunodiffusion or immunoelectrophoretic assay and the like. Plasma should be anti-coagulated before use, as is known in the art. Cellular elements and lipid may be removed from fluids, e.g., by centrifugation. For dilute fluids, such as urine, protein may be concentrated, e.g., by ultra-filtration or salting-out.

One preferred method of detecting and/or quantitating the novel antigen in fluid samples employs a competitive assay. An antibody immunoreactive with an epitope found on the novel antigen is attached to a solid support such as a polystyrene microtiter dish or nitrocellulose paper, using techniques known in the art. The solid support is then incubated in the presence of the fluid to be analyzed under conditions where antibody-antigen complexes form and are stable. Excess and unbound components of the fluid are removed and the solid support is washed so that antibody-antigen complexes are retained on the solid support. A fixed amount of a labeled polypeptide containing an epitope bound by the antibody attached to the solid support (i.e., an epitope of the novel antigen) is then incubated with the solid support. The polypeptide binds to any unbound antibody which is attached to the solid support. The polypeptide has been labeled by conjugation to a detectable moiety, such as biotin, peroxidase or radiolabel, by means well known in the art. Excess and unbound polypeptide is removed and the solid support is washed, as above. The detectable moiety attached to the solid support is quantitated. Since any cross-reactive protein in the sample and the polypeptide have competed for the same antibody binding sites, the cross-reactive protein in the fluid can be quantitated by its diminution of the binding of the polypeptide to the solid support.

The antibodies of the present invention can be used to detect epitopes found on the novel antigen in histological sections of ovarian cancer tissue as well as in other solid tumors such as, breast cancer, melanoma, etc. One can detect antibody binding to tissue sections by any detection means known in the art, for example, a radiolabel or a stain. A particularly useful stain employs peroxidase, hydrogen peroxide and a chromogenic substance such as aminoethyl carbazole. The peroxidase (a well known enzyme available from many sources) can be coupled to an antibody against the novel antigen or merely complexed via one or more antibodies to an antibody which specifically binds the novel antigen. For example, a goat anti-peroxidase antibody and a goat antibody against the novel antigen can be complexed via an anti-goat IgG. Such techniques are well known in the art. Other chromogenic substances and enzymes may also be used. Radiolabeling of antibodies may also be used to detect antibody binding to sections.

The precise technique by which expression of the novel antigen is detected in suspected ovarian cancer patients is not critical to the invention. Biochemical or immunological techniques can now be used which do not employ immunohistochemistry. Solution assay methods, including calorimetric, chemiluminescent or fluorescent immunoassays such as ELISA, sandwich and competitive immunoassays, immuno-diffusion, radio immunoassay, immunoelectrophoresis, Western blot and other techniques, may be used to detect and quantitate the novel antigen in a patient by preparing an extract of a tissue sample from the patient and assaying the extract.

Alternatively, any suitable immunoassay procedure may be used to detect the presence of autologous antibodies specific for the novel antigen in a biological sample from an individual. For example, a polypeptide encoded by the nucleic acid sequence of the novel antigen may be prepared by recombinant expression and immobilized on a microassay plate or other suitable surface. When the biological sample is incubated in the microassay plate or other suitable surface, antibodies specific for the novel antigen may be bound to the surface-immobilized antigen and detected by any of the means described above. Other methods for detecting specific antibodies in a biological sample will be readily apparent to the skilled person.

Although this assay has been described with particularity to detecting expression of the novel antigen of this invention, similar assays can also be used to detect expression of other protein analytes in biological fluids. For example, a detectably labeled polypeptide which shares an epitope with a protein analyte can be used to quantitate the analyte. A monospecific antibody is not required as the specificity is provided to the assay by means of the polypeptide.

Nucleotide Probe Assays for Expression

The nucleic acid probes described herein for use in screening gene libraries and selecting clones may also be used to detect mRNA transcripts in tumor cells that express the novel antigen of this invention. These probes preferably correspond to a sequence which encodes portions of the distinct sequences of the novel antigen (as deposited under ATCC accession No. [N]. The probe can be either single or double stranded DNA or RNA. The size of a probe can vary from less than approximately 20 nucleotides to hundreds of nucleotides. The most desirable nucleotide probes do not detect nucleotide sequences unrelated to their intended target, do not show significant homology with unrelated nucleotide sequences, and do not contain complementary sequences such that they would self-hybridize or fold upon themselves. The guanine and cytosine content of desirable probes is not so high as to promote non-specific hybridization with unrelated sequences rich in guanine and cytosine. Finally, the melting temperature and free energy of binding are generally favorably suited to the detection technique for which they are intended. The probe may be radio-labeled, labeled with a fluorescent material, a biotinylated nucleotide, or the like. Procedures for the preparation and labeling of nucleotide probes are well known in the art.

In situ hybridization of nucleotide probes to tissue sections is performed using standard methods, as described by, e.g., Baldino, et al., Methods in Enzymol., 1989, vol. 168, p. 761-77; Emson, et al., Methods in Enzymol., 1989, vol. 168, p. 753-61; Harper, et al., Methods in Enzymol., 1987, vol. 151, p. 539-51; Angerer, et al., Methods in Enzymol., 1987, vol. 152, p. 649-61; Wilcox, et al., Methods in Enzymol., 1986, vol. 124, p. 510-33, incorporated herein by reference, using nucleotide probes described herein. One preferred method for detecting mRNA associated with expression of novel antigen is in situ hybridization to tissue sections taken from tumors.

Alternatively, extracts of RNA from tissue samples can be analyzed for the presence of sequences encoding the proteins of this invention. The diagnostic test employing a nucleotide probe will employ a biological sample from an individual. Nucleic acids are recovered from the sample employing standard techniques well known to those skilled in the art. The nucleic acid may then be incubated with the probe and hybridization is thereafter detected. The presence of a nucleic acid whose sequence corresponds to that of the probe is preferably detected by Northern blot, or slot/dot blot.

Alternatively, a nucleic acid whose sequence corresponds to the sequence of of the novel antigen may be detected in the RNA extract of tumor tissue by nucleic acid amplification, using primers corresponding to the novel antigen's nucleic acid sequence, (see methods reviewed in Van Brunt, BioTechnology, 8:291-294, 1990). Similar primers can be used to amplify genomic DNA sequences encoding the novel antigen. The preferred method of amplification uses the polymerase chain reaction (PCR). Primers can be constructed corresponding to unique portions of the novel antigen nucleic acid sequence, determined as described above for nucleic acid probes. Using these primers, RNA or DNA in a nucleic acid extract of tumor tissue will be amplified by PCR only if it contains the unique sequences of the novel antigen.

An elevated level of the novel antigen mRNA in a cell corresponds to elevated expression of that antigen by the cell, and the novel antigen mRNA can be quantitated in a number of ways. Using Northern blotting, dot hybridization, ribonuclease protection assay or hybrid capture, purified RNA samples of known concentration and integrity can be hybridized with labeled probes. For each sample, the signal which is obtained can be compared ratiometrically to the signal obtained when the same sample is hybridized to a labelled probe for a constitutively expressed gene whose expression does not vary from cell to cell or sample to sample. Comparison of the ratios between different samples permits estimation of the differences in the novel antigen levels.

Alternatively, the level of the novel antigen mRNA expression can be estimated by in situ PCR or quantitative polymerase chain reaction. Using primers whose sequences correspond to the novel antigen nucleotide sequence, cDNA can be synthesized initially using reverse transcriptase, then the resultant cDNA amplified according to the polymerase chain reaction. The reaction is run under conditions and terminated so as to produce amounts of amplified products in proportion to the amount of MRNA originally present in the sample. The amount of product can be quantitated by ethidium fluorescence in comparison to known standards following electrophoresis, or by dot hybridization with labeled probes. Expression of constitutively expressed genes can be measured as a control, permitting standardized comparison of results, such as with the previously described hydridization reactions. Treatment of samples with ribonuclease A or other RNAses in control samples prior to amplification verifies that the signal is derived solely from RNA.

Elevated levels of a protein (e.g., the novel antigen) in a sample, such as the blood or other biological fluid, from a patient correlates with proliferation and likely metastasis of ovarian cancer as well as other solid tumors. Other tumors include lung cancer, genito-urinary tumors, and gastrointestinal tumors. The determination of elevated levels of the novel antigen is done relative to a healthy patient. This may be the same patient or a different patient. For example, a first sample may be collected immediately following surgical removal of a solid tumor. Subsequent samples may be taken to monitor recurrence of tumor growth and/or tumor cell proliferation and thereby provide prognostic information. Determination of the elevated level of cross-reactive protein may be done by direct detection of the protein or by indirect detection of its expression via measurement of MRNA encoding the protein. The detection may be in tissue sections by immunohistochemistry or in situ hybridization or it may be in extracts of tissue samples by solution immunoassay, or mRNA hybridization or amplification. In addition, the assay of the novel antigen in biological fluids can be used to distinguish between neoplastic and non-neoplastic fluid accumulations in patients carrying a malignant diagnosis.

Intracellular Tumor Antigens

The present inventors have discovered that cancer patients produce autologous antibodies to intracellular proteins expressed by the tumor cells. The mechanism for development of such an immune response is not known, although immunogenic stimulation may be due to release of the intracellular antigen by lysis of cells in necrotic tumor tissue. In any case, the unexpected presence of antibodies to tumor specific intracellular antigens provides a tumor specific marker that can be used diagnostically.

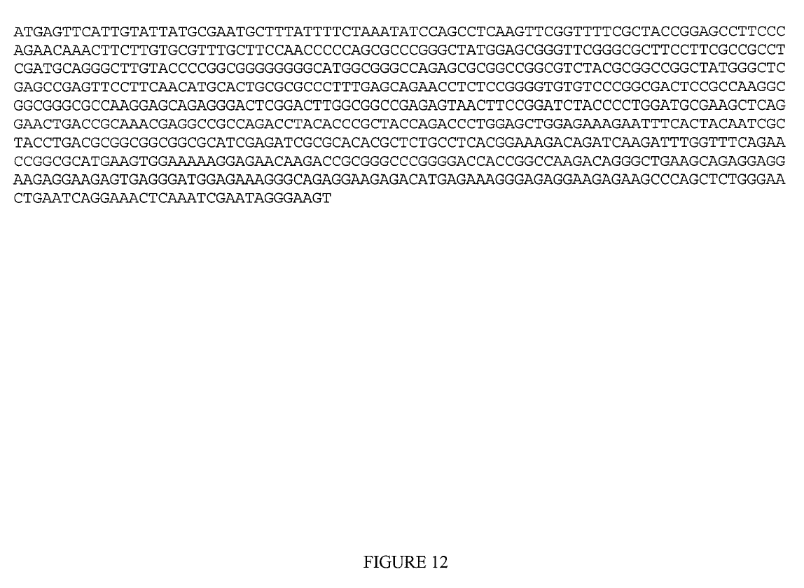

As described below in Example 4, the inventors have observed autologous antibodies to five specific intracellular antigens in the serum of patients with ovarian cancer: HOXA7, HOXB7, ABC-7 (ATP-binding iron transporter), ADP-ribosylation factor, and the novel protein associated with the gene sequence in clone 44B.1, deposited under ATCC accession No. [N]. This deposit has been made in accordance with the Budapest Treaty at the American Type Culture Collection (ATCC), 10801 University Boulevard, Manassas, Va. 20110-2209, USA (www.atcc.org), on Mar. 13, 2001. The inventors have also identified a previously unreported polymorph of HOXB7, which is detailed in FIG. 12 (SEQ ID NO: 1).

Based on the observation of autologous antibodies to these antigens in association with cancer, the present invention provides a screening method for use in identifying individuals which are likely to have cancer. A positive result to this screening assay will help support a diagnosis of cancer or at the very least justify additional diagnostic activity to confirm the likelihood suggested by the positive screening assay. In other words, determination that an individual has cells expressing one or more of these antigens, or has generated an immune response to one or more of these antigens, is consistent with the presence of neoplastic cells in the individual. Unless otherwise apparent from the context, reference herein to expression of an antigen is meant to include expression of variants of that antigen. The diagnostic likelihood is increased if an individual has cells expressing more than one of these antigens, and the likelihood increases further with increased number of the expressed antigens. In particular, the presence of one or more of these antigens lends support to a diagnosis of ovarian cancer. Detection of one or more of these antigens may also support diagnosis of breast cancer, melanoma, or endometrial and hematologic malignancies in the individual.

In particular, the inventors have discovered HOXA7 protein expression and HOXA7 antibodies in patients having a range of ovarian neoplasms including benign serous cystadenoma, micropapillary serous carcinoma, serous carcinoma, and endometrioid carcinoma (see Examples 5-7 and 9). Thus, detection of expression of HOXA7 and/or detection of HOXA7-specific antibody may alternatively support a diagnosis of ovarian neoplasia, including cancer or benign serous cystadenoma, and in an alternative mode, the present invention provides screening assays for these conditions.

Determination that cells are expressing one or more of these antigens may be made by any suitable assay technique. For example, tissue that is suspected of containing neoplastic cells may be subjected to immunohistochemistry using antibodies specific for one or more (or all five) of the antigens. Alternatively, MRNA associated with expression of one or more of the antigens may be detected by in situ hybridization using nucleic acid probes specific for the sequences of the antigens. In another alternative, lysates of cells in a sample of the tissue may be assayed immunologically for the presence of the antigens or tested for the presence of mRNA encoding any of the antigens. Immunoassays may use monoclonal or polyclonal antibodies specific for the antigens with appropriate controls, and assays for mRNA expression may use DNA or RNA probes or primer pairs specific for sequences encoding the antigens. Exemplary procedures for such assays are provided in the Examples below, and assays described in U.S. Pat. Nos. 5,734,022 and 5,759,791, incorporated herein by reference, may be used upon substitution of the appropriate specific antibodies or nucleic acids.

In a preferred mode of the screening assay of this invention, detection of antibodies to one or more of these five antigens in a sample of bodily fluid from an individual support the cancer diagnosis. Any suitable method of detecting the presence of antibodies specific for the indicated antigens may be used, including sandwich assays, competitive assays, ELISA, and other immunometric assays known in the art.

The screening assay also includes detection in extracellular fluid of one or more of the five antigens themselves. Typically this detection will be by immunoassay (sandwich, competitive, stacked antibody, etc.) or by functional assay based on the functional activity of the antigen.

Vaccines

Each of the intracellular tumor antigens discussed herein may be used as a sole vaccine candidate or in combination with one or more other antigens, the latter either being another of the intracellular antigens of this invention or another tumor antigen. Preferred are “cocktail” vaccines comprising two or more, more preferably three or more, even more preferably four or more, and most preferably all five intracellular tumor antigens. These vaccines may either be prophylactic (to prevent neoplastic developments) or therapeutic (to treat existing neoplasms).

Such vaccines comprise intracellular tumor antigen or antigens, usually in combination with “pharmaceutically acceptable carriers”, which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the antigen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc; (2) oil-in-water emulsion formulations (with or without other specific immunostimulating agents such as muramyl peptides (see below) or bacterial cell wall components), such as for example (a) MF59 (PCT Publ. No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) RibiTm adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox.TM.); (3) saponin adjuvants, such as Stimulon.TM. (Cambridge Bioscience, Worcester, Mass.) may be used or particles generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), etc; and (6) other substances that act as immunostimulating agents to enhance the effectiveness of the composition. Alum and MF59 are preferred.

As mentioned above, muramyl peptides include, but are not limited to, N-acetyl-muramyl-L-threonyl-D-isoglutamine (thr-MDP), N-acetyl-normuramyl-L-alanyl-D-iso-glutamine (nor-MDP), N-acetylmuramyl-L-alanyl-D-isoglutaminyl-L-alanine-2-(1′-2′-dipalmitoyl-sn -glycero-3-huydroxyphosphoryloxy)-ethylamine (MTP-PE), etc.

The immunogenic compositions (e.g., the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

Immunogenic compositions used as vaccines may comprise an immunologically effective amount of the antigenic polypeptides (i.e., the intracellular tumor antigens or antigenic fragments thereof), as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It may be expected that the amount will fall in a relatively broad range that can be determined through routine trials. Alternatively, the immunogenic compositions may comprise an expression vector encoding the antigenic polypeptide so that it is expressed in human cells. The immunogenic expression vector may be administered as naked DNA or incorporated into a viral gene-delivery vector or other suitable embodiment.

The immunogenic compositions are conventionally administered parenterally, e.g., by injection, either subcutaneously or intramuscularly. Additional formulations suitable for other modes of administration include oral and pulmonary formulations, suppositories, and transdermal applications. Dosage treatment may be a single dose schedule or a multiple dose schedule. The vaccine may be administered in conjunction with other immunoregulatory agents.

Drug Screening Methods

Diagnosis may not be the only useful property associated with anomalous expression of these intracellular antigens in tumor cells. Expression of these intracelluar antigens may be mechanistically associated with cancer. One consequence is that these antigens may serve as therapeutic targets. This invention provides screening methods for therapeutically active materials based on the antigens identified herein.

Drug screening procedures according to this invention utilize a test system that includes a screening cell which expresses at least one antigen identified herein (“the test antigen”). The screening cell may be a recombinant cell containing a nucleic acid sequence encoding the antigen in condition for expression of the test antigen. Alternatively, the screening cell may be a cell from a tumor cell line which overexpresses the test antigen. Assaying cell lines for overexpression of one or more antigens according to this invention is a routine matter for the skilled person in view of the present disclosure.

Test compounds are screened by incubation of the screening cell in the presence and absence of the test compound, and monitoring the level of biological activity associated with the test antigen. For example, if the test antigen is the ABC7 ATP-dependent iron transporter (consistent with the sequence of gb|AF1336591AF|33659), the mitochondrial iron transporter activity is monitored. If the test antigen is ADP-ribosylation factor-1 (Arf-1), the screening cell is monitored for vesicular transport (see Mandiyan, V., Andreev, J., Schlessinger, J., and Hubbard, S. R., “Crystal Structure of the ARF-GAP Domain and Ankyrin Repeats of PYK2-Associated Protein Beta,” Embo. J., 18:6890-6898, 1999). As discussed above, Arf-1 cycles between the cytosol and membrane depending upon its nucleotide status, and membrane association occurs through conversion of ARF-1 to the active GTP-bound form by guanine nucleotide exchange factors. Once membrane bound, ARF-1 participates in recruitment of coatomer proteins that are required for budding and fission of membranes. (A coated vesicle must be uncoated before fusion with the acceptor compartment can occur, and uncoating requires hydrolysis of GTP to GDP, a process dependent upon interaction of Arf-1 with GTPase-activating proteins.) ARF-1 is necessary for maintenance of both golgi and endosome structure (Gaynor, E. C., Chen, C. Y., Emr, S. D., and Graham, T. R., “ARF is Required for Maintenance of Yeast Golgi and Endosome Structure and Function,” Mol. Biol. Cell, 9:653-670, 1998), Arf-1 plays a role in Phospholipase D (PLD) signaling (Brown, H. A., Gutowski, S., Moomaw, C. R., Slaughter, C., and Steinweis, P. C., “ADP-Ribosylation Factor, a Small GTP-Dependent Regulatory Protein, Stimulates Phospholipase D Activity,” Cell, 75:1137-1144, 1993), and Arf-1 also interacts with the second messenger phosphatidylinositol 4,5-bisphosphate (Randazzo, P. A., “Functional Interaction of ADP-Ribosylation Factor I With Phosphatidylinositol 4,5-bisphosphate,” J. Biol. Chem., 272:7688-7692, 1997; Randazzo, P. A., Terui, T., Sturch, S., Fales, H. M., Ferrige, A. G., and Kahn, R. A., “The Myristoylated Amino Terminus of ADP-Ribosylation Factor I is a Phospholipid- and GTP-Sensitive Switch,” J. Biol. Chem., 270:14809-14815, 1995). The screening cell may be monitored for internal vessicle structure, for elevation of PLD activity, GTPase activity, and/or for activation of systems dependent on phosphatidylinositol 4,5-bisphosphate. Test compounds that alter one or more of these activities relative to control cells are noted for further testing. If the test antigen is HOXA7 and/or HOXB7, members of the HOX family of homeobox genes which encode transcription factors that regulate normal cellular proliferation and differentiation during development (Mark, M., Rijli, F. M. and Chambon, P., Pediatr. Res., 42:421-429, 1977; Cillo, C., Faiella, A., Cantile, M. and Boncinelli, E., Exp. Cell. Res. 248:1-9, 1999), the screening cell may be a hematopoetic cell monitored for control of hematopoiesis or for transforming activity. Expression of HOXB7 constitutively activates basic fibroblast growth factor (bFGF) expression through one of the five putative homeodomain binding sites present in its promoter (Care, A., Silvani, A., Meccia, E., Mattia, G., Stoppacciaro, A., Parmiani, G., Peschle, C., and Colombo, M. P., “HOXB7 Constitutively Activates Basic Fibroblast Growth Factor in Melanomas,” Mol. Cell Biol., 16:4842-4851, 1996), and the effect of the test compound on expression of bFGF may be monitored.

Test compounds which affect one or more of the screening cell systems have an increased likelihood to have effects on tumor cells. Typically, such compounds will be further tested by evaluating their effect in cell growth models or cytotoxicity test systems to confirm the effect on tumor cells. Alternatively, the anti-tumor properties of selected test compounds may be evaluated by administering the compounds to test animals which have tumors, either as grafts or because the animals are bred for a propensity to develop tumors. Suitable systems for such confirmatory testing are well known in the art.

### EXAMPLES

The examples presented below are provided as a further guide to the practitioner of ordinary skill in the art and are not to be construed as limiting the invention in any way.

### Example 1

### Detection of Autologous Antibody to Ovarian Cancer by Western Blot

Expression of eukaryotic cDNAs in bacteria often does not generate proteins in their native conformation. Cellular proteins subjected to SDS-PAGE and Western blot are generally denatured. Therefore those antibodies to the linear epitopes relevant for screening proteins expressed in bacteria will often be detected by Western blot.

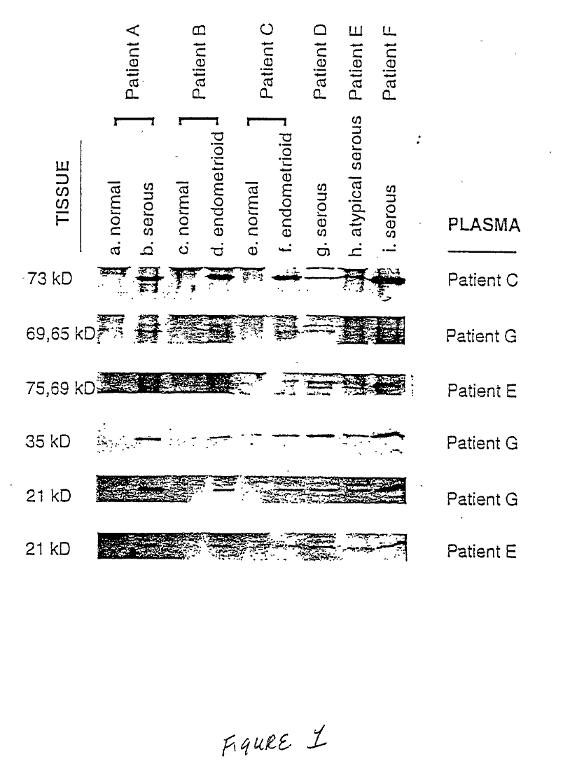

To initiate this study, patient sera were screened for autologous antibodies to ovarian tumor antigens by Western blot. Twenty micrograms of tissue extract was loaded in each lane and equal loading confirmed by Ponceau S staining. 20 jig of protein extracted from tissue specimens including normal ovary (a,c,e), and ovarian tumors of serous (b,g,i), atypical serous (h) and endometrioid (d,f) histology were separated on a 10% SDS-PAGE gel and transferred to a PVDF membrane. After electroblotting, membranes were probed with patient sera at 1:500 and then 1:10,000 peroxidase-linked anti-human immunoglobulin antibody (FIG. 1). The membranes were probed with a 1:500 dilution of serum derived from the same patient, or a different ovarian cancer patient (see letter coding) and 1:10,000 peroxidase linked anti-human immunoglobulin secondary antibody. The blots were developed using a chemiluminescent substrate and the molecular weights of protein bands specific for tumor tissue determined (indicated on the left-hand side).

Since human tissue extracts were probed in these Western blots, proteins likely containing immunoglobulin-like domains were detected by the secondary antibody (peroxidase-linked anti-human immunoglobulin). However, several antigens (estimated molecular weights 21 kDa, 35 kDa, 65 kDa, 69 kDa, 73 kDa, and 75 kDa) were present in tumor extracts but absent from normal ovarian tissue from the same patient. None of these antigens were recognized by serum antibody from a healthy volunteer and secondary antibody (not shown). The antigens appear to be tumor-specific, although their absence from other tissues remains to be confirmed. Most importantly, a subset of these antigens (molecular weights 21 kDa, 35 kDa, 73 kDa) were expressed in all seven ovarian tumor extracts and were not detected, or were at significantly lower levels in three normal ovarian tissues (FIG. 1).

The 73 kDa protein detected with sera from patient C (FIG. 1) may correspond to the 71 kDa glucose-regulated protein 78 previously described as containing epitopes specific to ovarian cancer (Chinni et al., 1997). The 75, 69, 65, 35 and 21 kDa tumor antigens (FIG. 1) identified by these sera appear novel. However, previous studies have identified antigens defined by antibodies recovered from complexes associated with epithelial ovarian cancer. Two ovarian tumor specific components of 64 kDa and 23 kDa were identified by Stolbach et al., and perhaps correspond to the 65 kDa and 21 kDa autoantigens in FIG. 1 (Dawson et al., 1983). Using autologous antibody Lutz and Dawson immunoprecipitated tumor antigens of 49, 46, 33 and 25 kDa from membranes of ovarian tumor (Lutz, P. M. and Dawson, J. R., “Activity of Antibodies Recovered from Immune Complexes of Ovarian Cancer Patients,” Cancer Immunol. Immunother., 17:180-189, 1984), and the 33 kDa antigen may correspond to the 35 kDa antigen in FIG. 1. Therefore some of the tumor antigens are common to ovarian tumors, even those of distinct phenotype. Furthermore, the 21 kDa tumor antigen was detected by antibodies derived from two patients (FIG. 1, patients E and G). Therefore, in a very limited screen of ovarian cancer patient serum samples, we have already identified a number of candidate tumor antigens that appear commonly recognized by high titer autologous antibodies.

### Example 2

### cDNA Expression Library Generated from Cell Line OV1063

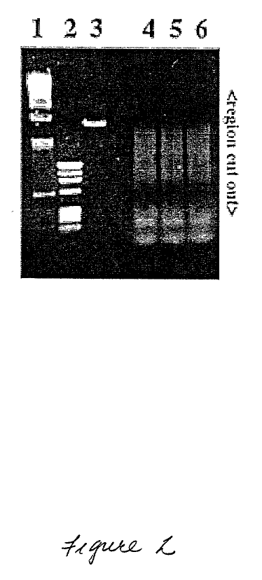

We have generated a cDNA expression library in λZAP bacteriophage from polyA⁺ mRNA purified from the cancer cell line OV1063 (Horowitz, A. T., Treves, A. J., Voss, R., Okon, E., Fuks, Z., Davidson, L., and Biran, S., “A New Human Ovarian Carcinoma Cell Line: Establishment and Analysis of Tumor-Associated Markers,” Oncology, 42:332-337, 1985). OV1063 cells generate tumors in immunodeficient mice that have been widely used for drug therapy studies (Miyazaki, M., Schally, A. V., Nagy, A., Lamharzi, N., Halmos, G., Szepeshazi, Kr., and Armatis, P., “Targeted Cytotoxic Analog of Luteinizing Hormone-Releasing Hormone AN-207 Inhibits Growth of OV-1063 Human Epithelial Ovarian Cancers in Nude Mice,” Am. J. Obstet. Gynecol., 180:1095-1103, 1999). A cell line rather than tumor tissue was used to reduce problems in screening caused by contaminating lymphocytes (as the secondary antibody used in screening will recognize cloned human immunoglobulin genes). Specimens of solid tumors or malignant ascites comprise mixed cell populations. The use of heterogeneous source material for library construction will skew the profile of recombinantly expressed proteins leading to under representation and possible loss of detection of the sought antigen. Thus, despite potential for tissue culture artifacts, a cell line was used to generate a cDNA library likely to provide appropriate clonal representation of ovarian cancer antigens. Furthermore patient sera demonstrated good reactivity with OV1063 cell lysates by Western blots (not shown). Total RNA was isolated by the guanidinium isothiocyanate method from OV1063. Poly(A)⁺ mRNA was purified by hybridization to and subsequent detachment from oligo(dT) cellulose. First strand cDNA synthesis was performed using an oligo(dT) primer containing a Xho I site. Following second strand synthesis, Eco RI adapters were ligated. cDNAs of >0.3 kb were gel-purified from the free linkers by agarose gel electrophoresis (see FIG. 2), digested with Aho I and cloned directionally into Eco RI/Aho I sites of the λZAP-Express vector (Stratagene). Lane 1 contains λ/HindIII markers, lane 2 contains φX174/HaeIII markers, lane 3 contains EcoRI,XhoI digested pBK-CMV plasmid and lanes 4-6 contain linker-ligated OV1063 cDNA. Ligated cDNA were packaged into phage particles in vitro. Upon packaging of recombinants, the bacteriophage were titered by infection of E. coli strain XL1-Blue MRF′. 800,000 independent clones were generated. The primary library was subsequently amplified for screening. The average size of cDNA inserts was approximately 1 kb indicating the integrity of the OV1063 RNA and cDNA synthesis.

### Example 3

### Screening of Expression Library with Ovarian Cancer Patient Serum

An important issue in screening of λZAP bacteriophage expression libraries with patient serum is the elimination of non-specific antibody i.e. antibody specific for E.coli. We have eliminated this non-specific reactivity by confluent infection of E.coli lawns with wild type λZAP bacteriophage to achieve confluent lysis and subsequent transfer to nitrocellulose filters. Patient sera are diluted in blocking buffer and preabsorbed overnight with such a filter. This procedure permits screening of the λ expression library for specifically immuno-reactive clones.

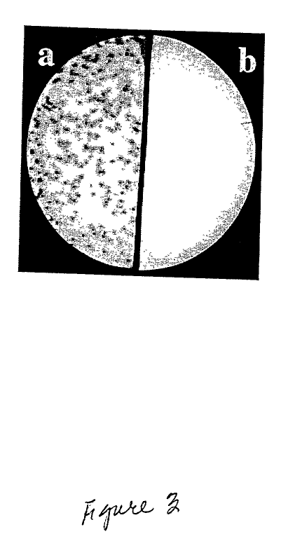

Serum from a particular patient with serous ovarian carcinoma demonstrated strong reactivity with ovarian tumors, and the OV1063 cell lysates, by Western blot. Therefore, the OV1063 CDNA expression library of 800,000 independent clones in λZAP bacteriophage was screened using this particular ovarian cancer patient serum. Expression of proteins was induced with IPTG in E. coli infected with recombinant λphage, and transferred on to nitrocellulose membranes. Membranes were incubated with diluted patient serum (1:500) which had been preabsorbed with wild-type phage-infected E.coli lysate to remove non-specific reactivity. After washing, membranes were incubated with alkaline phosphatase-conjugated anti-human IgG antibody and positive plaques visualized by reactivity with 5-bromo-4-chloro-indolyl-phosphate/nitroblue tetrazolium (BCIP/NBT). Positive phage plaques identified in the first round of screening were verified and purified to monoclonality by repeated rounds of immuno-screening. False-positive plaques were eliminated by screening with secondary antibody alone. Verification of positive phage clones is demonstrated in FIG. 3 for a single clone isolated by primary library screening. Recombinants from a single clone were transferred to an IPTG-soaked membrane, one half of which was incubated with patient serum diluted 1:500 (panel a) or buffer alone (panel b). Reactivity with expressed serum antibodies was detected by alkaline phosphatase conjugated antihuman IgG antibody. Phagemids were excised by co-infection of E. coli with purified positive phage and filamentous helper phage. To date 31 positive ‘SEREX clones’ that do not react with secondary antibody alone, have been isolated.

The cDNA were excised from recombinant λZAP bacteriophage by co-infection of E. coli strain XL1 -Blue with Exassist Interference resistant helper phage (Stratagene). In this way the cDNA are transferred 3′ to both E. coli and mammalian promoters in the phagemid vector pBK-CMV in E. coli XLOLR.

### Example 4

### Identity of OV1063 Antigens Recognized by Ovarian Cancer Patient Serum

DNA preparations of clones have been sequenced. The sequences were compared to known sequences in Genbank using BLAST searches. Five independent species have been identified among the 31 cDNA clones sequenced to date as summarized in Table 1. The novel sequence mentioned in Table 1 occurs as a fragment released by EcoRI/XhoI digestion of Clone 44B.1, deposited under ATCC accession No. [N].

TABLE 1
Identity of clones isolated in a screen of an OV1063 cDNA expression
library with sera of an ovarian serous carcinoma patient.

| Identity of clone | Genbank sequence | # clones |
|---|---|---|
| HOXA7 | AF026397 | 21 |
| HOXB7 | NM004502 | 7 |
| ABC7 (ATP-binding iron transporter) | AF133659 | 1 |
| ADP-ribosylation factor | AF052179 | 1 |
| Novel sequence (clone 44B.1) | No match | 1 |

One gene isolated encodes a full-length cDNA clone of the GTP-binding protein human ADP-ribosylation factor-i (consistent with the sequence of gb|M84326.1|LHUMADPRFIA) and another encodes the ABC7 ATP-dependent iron transporter (consistent with the sequence of gb|AF133659|AF133659). The ABC7 gene product is a putative mitochondrial iron transporter gene that is mutated in X- linked sideroblastic anemia and ataxia (XLSA/A) (Allikmets, R., Raskind, W. H., Hutchinson, A., Schueck, N. D., Dean, M., and Koeller, D. M., “Mutation of a Putative Mitochondrial Iron Transporter Gene (ABC7) in X-Linked Sideroblastic Anemia and Ataxia (XLSA/A),” Hum. Mol. Gent., 8:743-749, 1999). ADP-ribosylation factor-1 (Arf-1) is a small G protein involved in vesicular transport (Mandiyan et al., 1999). Arf-1 cycles between the cytosol and membrane depending upon its nucleotide status. Membrane association occurs through conversion of ARF-1 to the active GTP-bound form by guanine nucleotide exchange factors. Once membrane bound, ARF-1 participates in recruitment of coatomer proteins that are required for budding and fission of membranes. A coated vesicle must be uncoated before fuision with the acceptor compartment can occur. Uncoating requires hydrolysis of GTP to GDP, a process dependent upon interaction of Arf-1 with GTPase-activating proteins. Mutations of the ARF-1 ortholog in yeast demonstrate that this gene is necessary for maintenance of both golgi and endosome structure (Gaynor et al., 1998). Furthermore, Arf-1 plays a role in Phospholipase D (PLD) signaling (Brown et al., 1993) and also interacts with the second messenger phosphatidylinositol 4,5-bisphosphate (Randazzo, 1997; Randazzo et al., 1995). Arf-dependent PLD activity is elevated in experimental tumors in rats (Yoshida, M., Okamura, S., Kodaki, T., Mori, M., and Yamashita, S., “Enhanced Levels of Oleate-Dependent and Arf-Dependent Phospholipase D Isoforms in experimental Colon Cancer,” Oncol. Res., 10:399-406, 1998). Interestingly, lysophosphatidic acid (LPA) both stimulates the growth of ovarian cancer cells, and activates PLD (Eder, AM., Sasagawa, T., Mao, M., Aoki, J., and Mills, G. B., onstitutive and Lysophosphatidic Acid (LPA)-Induced LPA Production: Role of Phospholipase D and Phospholipase A2, “Clin. Cancer Res., 6:2482-2491, 2000; Fang, X., Gaudette, D., Furui, T., Mao, M., Estrella, V., Eder, A., Pustilnik, T., Sasagawa, T., Lapushin, R., Yu, S., Jaffe, R.B., Wiener, J. R., Erickson, J. R., and Mills, G. B., “Lysophospholipid Growth Factors in the Initiation, progression, Metastases, and Management of Ovarian Cancer,” Ann. N.Y Acad. Sci., 905:188-208, 2000).

Of significant interest are findings that twenty-one clones encode full length human HOXA7, while another seven encode human HOXB7. Coding sequences of the HOXB7 clones were identical to the original published sequence (GenBank No. NM004502), except for two nucleotide substitutions GC and GA which respectively altered residue 53 from gly to ala and residue 173 from ala to thr. One or another of these substitutions were also present in other published HOXB7 clones ((Chariot, A., Princen, F., Gielen, J., Merville, M. P., Franzoso, G., Brown, K., Siebenlist, U., and Bours, V., J. Biol. Chem., 274:5318-5325, 1999), GenBank No. XM008559). HOXA7 and HOXB7 are members of the HOX family of homeobox genes which encode transcription factors that regulate normal cellular proliferation and differentiation during development (Mark, et al, 1997; Cillo, et al., 1999). HOX genes have been well-studied in their control of hematopoiesis, and their aberrant expression in leukemias and other cancers has implicated their involvement in tumorigenesis (Cillo, et al., 1999; Van Oostveen, J. W., Bijl, J. J., Raaphorst, F. H., Walboomers, J. J. M., and Meijer, C. J. L. M., Leukemia, 13:1675-1690, 1999; Cillo, C., Invasion Metastasis, 14:38-49, 1995). The double knockout of these paralogous genes yields first and second rib defects (Chen, F., Greer, J., and Capecchi, M. R., “Analysis of HOXA7/HOXB7 Mutants Suggests Periodicity in the Generation of the Different Sets of Vertebrae,” Mech. Dev., 77:49-57, 1998). These transcription factors have been demonstrated in vitro and in mouse models to have transforming ability, which also strongly implicates their role in tumorigenesis (Chang, P. Y., Kozono, T., Chida, K., Kuroki, T., and Huh, N., “Differential Expression of HOX Genes in Multistage Carcinogenesis of Mouse Skin,” Biochem. Biophys. Res. Commun., 248:749-452, 1998; Maulbecker, C. C., and Gruss, P., “The Oncogenic Potential of Deregulated Homeobox Genes,”, Cell Growth Differ., 4:431-441, 1993). HOXB7 transcription is silent in normal quiescent melanocytes, but becomes highly active in melanomas (Care et al., 1996). Furthermore, expression of HOXB7 constitutively activates basic fibroblast growth factor (bFGF) expression through one of the five putative homeodomain binding sites present in its promoter (Care et al., 1996). Retrovirally transduced HOXB7 potently stimulates proliferation of hematopoietic progenitor/stem cells from human adult peripheral blood, suggested to represent a preleukemic immortalization step (Care, A., Valtieri, M., Mattia, G., Meccia, E., Masella, B., Luchetti, L., Felicetti, F., Colombo, M. P., and Peschle, C., “Enforced Expression of HOXB7 Promotes Hematopoietic Stem Cell Proliferation and Myeloid-Restricted Progenitor Differentiation,” Oncogene, 18:1993-2001, 1999). Indeed, transduction of the SkBr3 breast carcinoma line with the HOXB7 gene also induces bFGF expression, thus increasing cell proliferation and reducing growth factor dependence (Care et al., 1998). bFGF has both angiogenic and mitogenic properties, and therefore autocrine stimulation may contribute to the rapid growth of ovarian epithelial carcinoma (Di Blasio, A. M., Carniti, C., Vigano, P., and Vignali, M., “Basic Fibroblast Growth Factor and Ovarian Cancer,” J. Steroid Biochem. Mol. Biol, 53:375-379, 1995). Elevated levels of bFGF have been identified in both tumor tissue (Fujimoto, J., Ichigo, S., Hori, M., Hirose, R., Sakaguchi, H., and Tamaya, T., “Expression of Basic Fibroblast Growth Factor and tis MRNA in Advanced Ovarian Cancers, Eur. J Gynaecol. Oncol., 18:349-352, 1997; Obermair, A., Speiser, P., Reisenberger, K., Ullrich, R., Czerwenka, K., Kaider, A., Zeillinger, R., and Miksche, M., “Influence of Intratumoral Basic Fibroblast Growth Factor Concentration on Survival in Ovarian Cancer Patients,” Cancer Lett., 130:69-76, 1998) and pre-treatment serum (Barton, D. P., Cai, A., Wendt, K., Young, M., Gamero, A., and De Cesare, S., “Angiogenic Protein Expression in Advanced Epithelial Ovarian Cancer,” Clin. Cancer Res., 3:1579-1586, 1997; Dirix, L.Y., Vermeulen, P. B., Pawinski, A., Prove, A., Benoy, I., De Pooter, C., Martin, M., and Van Oosterom, A. T., “Elevated Levels of the Angiogenic Cytokines Basic Fibroblast Growth Factor and Vascular Endothelial Growth Factor in Sera of Cancer Patients,” Br. J. Cancer, 76:238-243, 1997). Although bFGF levels in cyst fluid did not correlate significantly with malignancy (Hazelton, D., Nicosia, R. F., and Nicosia, S. V., “Vascular Endothelial Growth Factor Levels in Ovarian Cyst Fluid Correlate with Malignancy,” Clin. Cancer Res., 5:823-829, 1999), addition of bFGF to cultured ovarian cancers cells induces proliferation (Crickard, K., Gross, J. L., Crickard, U., Yoonessi, M., Lele, S., Herblin, W. F., and Eidsvoog, K., “Basic Fibroblast Growth Factor and Receptor Expression in Human Ovarian Cancer,” Gynecol Oncol., 55:277-284, 1994). Therefore, the finding that HOXA7 and HOXB7 are immnunogenic ovarian tumor antigens is highly provocative (Care et al., 1996; Chang et al., 1998; Perkins, A., Kongsuwan, K., Visvader, J., Adams, J. M., and Cory, S., “Homeobox Gene Expression Plus Autocrine Growth Factor Production Elicits Myeloid Leukemia,” Proc. Natl. Acad. Sci., USA, 87:8398-8402, 1990; Srebrow, A., Friedmann, Y., Ravanpay, A., Daniel, C. W., and Bissel, M. J., “Expression of HOXA-1 and HOXB-7 is Regulated by Extracellular Matrix-Dependent Signals in Mammary Epithelial Cells,” J. “ell Biochem., 69:377-391, 1998).

### Example 5

### Analysis of SEREX antigen expression in normal ovarian epithelium and ovarian tumors.

The high degree of sequence conservation and low level of mRNA expression exhibited by the HOX genes prohibit the use of Northern blot methodology to analyze mRNA transcript level. Rather, RT-PCR based methodology, as described in Alami et al. (1999), was used (Alami, Y., Castronovo, V., Belotti, D., Flagiello, D., and Clausse, N., “Hoxc5 AND hoxc8 Expression are Selectively Turned on in Human Cervical Cancer Cells Compared to Normal Keatinocytes,” Bioche. Biophys. Res. Commun., 257:738-745, 1999). The following conditions were used to PCR amplify HOXA7: primers GAGCTGGAGAAGGAGTTCCA (SEQ ID NO: 2) and CTTTCTTCCACTTCATACGA (SEQ ID NO: 3) for 40 cycles, producing a 134bp product. For HOXB7 primer pair AGAGTAACTTCCGGATCTA (SEQ ID NO: 4) and TCTGCTTCAGCCCTGTCTT (SEQ ID NO: 5) was used to amplify for 35 cycles a 274 bp product. HOXA7 and B7 transcript levels were compared by semi-quantitative RT-PCR to β-actin expression in cancer lines OV1063 and OVCAR-3, and serous, endometrioid, and poorly differentiated ovarian cancers tumors and ovarian surface epithelial cells (OSE) scraped from normal ovary.

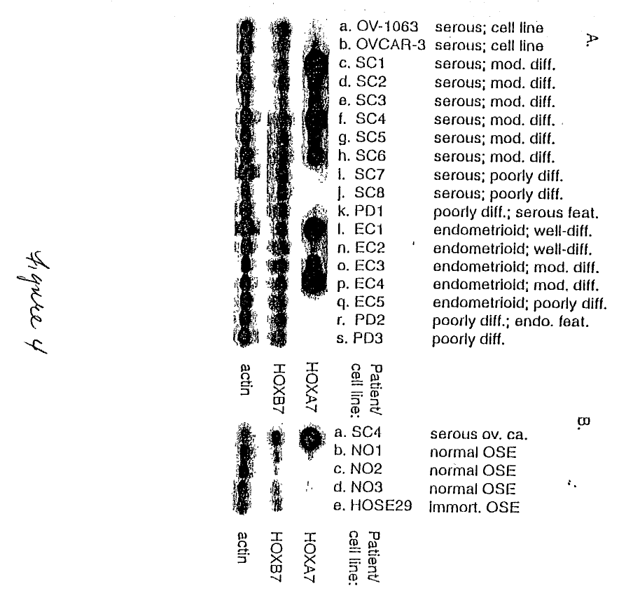

FIG. 4 shows representative data from our RT-PCR analysis of HOX gene expression in ovarian tumors and normal ovarian surface epithelium. The data suggest that HOXA7 mRNA is not expressed at significant levels in normal ovarian surface epithelium (FIG. 4B, lanes b-d). HOXA7 transcript expression is low in cell lines derived from serous ovarian carcinomas, and OV1063 cells from which our cDNA library was derived (FIG. 4A, lanes a and b). Moderately-to-well differentiated serous and endometrioid carcinoma express HOXA7, but poorly differentiated carcinoma express little or no HOXA7 in our limited analysis. The low expression of HOXA7 in cell lines derived from serous ovarian carcinoma is consistent with their poorly differentiated phenotype as xenografts in immunodeficient RAG-1 knockout mice (data not shown).

HOXB7 expression was determined by semi-quantitative RT-PCR in normal OSE and in IOSE-29 cells, a non-tumorigenic cell line established by immortalizing normal OSE cells with SV40 large T antigen (Maines-Bandiera, S. L., Kruk, P. A. and Auersperg, N., Am. J. Obstet. Gynecol., 167:729-735, 1992). Reverse transcription was performed using 1 μg of DNase I-treated total RNA, 500 ng of oligo(dT) and Superscript II reverse transcriptase (Life Technologies). Amplification of cDNAs for HOXB7 and for β-actin were performed as described by others (Alami, Y., Castronovo, V., Belotti, D., Flagiello, D. and Clausse, N., Biochem. Biophys. Res. Comm., 257:738-745, 1999; Nakajima-Iijima, S., Hamada, H., Reddy, P. and Kakunaga, T., Proc. Natl. Acad. Sci. USA, 82:6133-6137, 1985) using Platinum Taq DNA polymerase (Life Technologies). Briefly, amplification was performed with a 2 min start at 94° C., denaturation at 94° C. for 1 min, annealing at 55° C. for 1 min and extension at 72° C. for 1 min, for 35 cycles for HOXB7 and 25 cycles for 13-actin. Titrations were performed to ensure a linear range of amplification. Primers were the same as used by others (Alami, et al., 1999; and Nakajima-lijima, et al., 1985) and were as follows: for HOXB7 5′AGAGTAACTTCCGGATCTA-3′ (SEQ ID NO: 4) and 5′-TCTGCTTCAGCCCTGTCTT-3′ (SEQ ID NO: 5), and for β-actin 5′-ATGATATCGCCGCGCTCG-3′ (SEQ ID NO: 6) and 5′-CGCTCGGTGAGGATCTTCA-3′ (SEQ ID NO: 7). Southern blot analysis of RT-PCR products was conducted using ³²P-labelled β-actin cDNA (Clontech, Palo Alto, Calif.) and HOXB7 cDNA. Hybridization signals were quantified by PhosphorImager analysis (Molecular Dynamics, Sunnyvale, Calif.).

Shown in FIG. 4 are Southern blots of HOXB7 and β-actin RT-PCR products in OV-1063, OVCAR-3 and IOSE-29 cells (FIG. 4A, lanes a,b; FIG. 4B, lane e), specimens of normal OSE (FIG. 4B, lanes b-d) and ovarian carcinomas (FIG. 4A, lanes c-s). Histology of carcinomas ranged from poorly differentiated (diff.) with either serous or endometrioid features (feat.) to moderately (mod.) and well-differentiated serous and endometrioid. The specimen used for analysis shown in FIG. 4B, lane a is the same as that in FIG. 4A, lane f. Low levels of HOXB7 expression were detected by semi-quantitative RT-PCR analysis in normal OSE and in IOSE-29 cells (FIG. 4). However, markedly higher levels of HOXB7 expression were detected in primary ovarian carcinomas. Such elevated levels were consistent between specimens of carcinomas which varied widely in their degree and type of histologic differentiation, and also in stage of disease. OV-1063 cells and the ovarian carcinoma cell line OVCAR-3 also expressed HOXB7 at levels similar to those in tumor tissue specimens (FIG. 4). These observations indicate that elevation of HOXB7 expression is a common feature of ovarian carcinomas and may render HOXB7 immunogenic.

Expression of HOXB7 MRNA has a different pattern to that of HOXA7. All ovarian carcinomas and including cell lines OV1063 and OVCAR-3 express HOXB7 mRNA at levels higher than normal ovarian epithelium (FIG. 4). HOXB7 mRNA expression is also low in IOSE 29 cells, a non-tumorigenic cell line established by immortalization of normal ovarian surface epithelium cells with SV40 large T antigen.

### Example 6

### Development of an Immunohistochemical Stain for HOXA7.

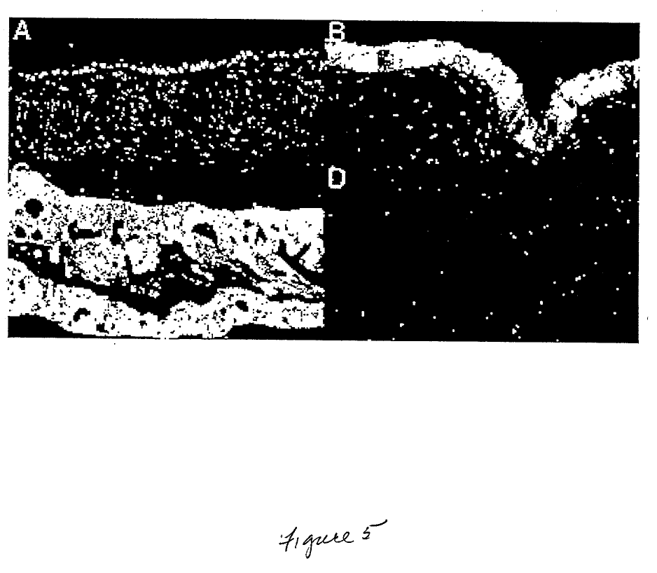

Since the cDNA species most frequently identified in our screen of the OV1063 cDNA expression library with patient serum encoded HOXA7, we chose to develop an immunohistochemical stain in tissue using rabbit antiserum to a peptide (DKADEGVLHGPAEA, SEQ ID NO: 8) that is specific to HOXA7 (Berkeley Antibody Company, USA). Immunohistochemical staining of 5 μm tissue sections was performed using rabbit antiserum to a peptide (DKADEGVLHGPAEA, SEQ ID NO: 8) that is specific to HOXA7 (Berkeley Antibody Company, USA) at 1:500, and the results are shown in FIG. 5. Panel A shows a stained section of normal ovary, including surface epithelium, Panel B is a stained section of serous ovarian cystadenoma, Panel C is a stained section of moderately-differentiated serous ovarian carcinoma demonstrated positive for HOXA7 transcript by RT-PCR, and Panel D is a representative stained section of poorly-differentiated ovarian carcinoma.

As can be seen in FIG. 5, the HOXA7 peptide antibody reacted strongly with the epithelial tumor, but not its stromal component. Only minimal background staining was observed with secondary antibody alone (not shown). Correlation of in situ histochemistry and mRNA levels is essential for rigorous analysis of HOXA7 expression in normal and tumor tissue. Immuno-staining studies confirm high HOXA7 staining in serous and endometrioid, but not poorly differentiated ovarian carcinoma or normal ovarian surface epithelium.

### Example 7

### Analysis of Autologous Antibody Responses to HOX Proteins

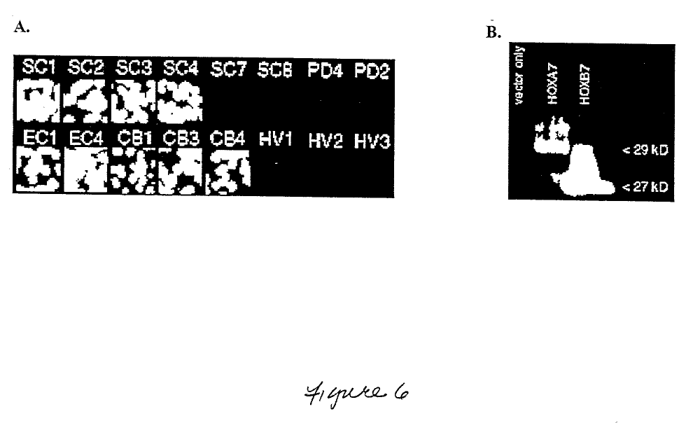

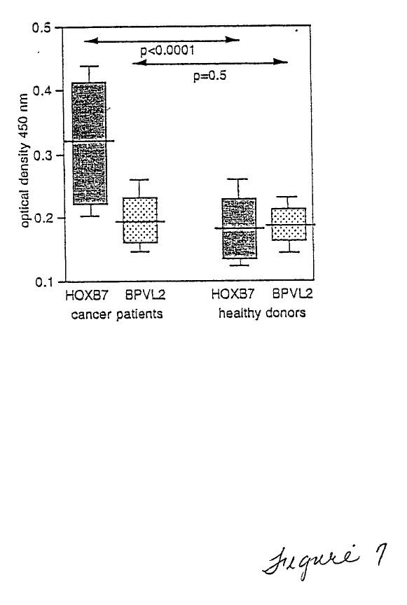

Since the majority of clones isolated encoded HOXA7, a small pilot experiment was performed to examine the frequency with which ovarian cancer patients generate antibody specific for HOXA7. Lawns of E.coli were infected with a 1:5 ratio of recombinant λ bacteriophage containing the HOXA7 gene and non-reactive X bacteriophage at an MOI sufficient to produce sub-confluent lysis in 24h. The plaques were transferred to nitrocellulose soaked in IPTG and protein expression induced for 5 h. The filters were lifted, cut into slices. After blocking, the slices were incubated with pre-cleared serum from 8 patients and 3 controls. Reactivity of each serum was detected in parallel using AP-anti-human Ig and BCIP/NBT development. The membrane slices were immediately photographed (see FIG. 6A). Sera of patients with moderately differentiated serous ovarian carcinoma (SC1-4), endometrioid ovarian carcinoma (well-differentiated EC1, or moderately differentiated EC4) or serous ovarian cystadenoma (CB1,3,4) reacted specifically with HOXA7-expressing bacteriophage plaques. Sera from patients with poorly differentiated serous ovarian carcinoma (SC7,8) or poorly differentiated ovarian carcinoma (with serous features, PD4 or endometrioid features, PD2) and of healthy volunteers (HV1-3) failed to react (background levels) with HOXA7. The data suggest that sera of patients with histologically differentiated ovarian tumors are more reactive than control sera.

To confirm reactivity, the HOX gene products were expressed in bacteria. Bacterial lysates were probed by Western blot with the patient serum used to screen the cDNA library at 1:500 (FIG. 6B), demonstrating specific reactivity of this patient serum with recombinant human HOXA7 and B7. A Western blot was performed using crude extracts of E.coli transformed with pBK-CMV vector alone, or HOXA7 or HOXB7 cDNAs cloned into pBK-CMV and expression of β-galactosidase fusion proteins induced with 1 mM IPTG for 5 h. After separation on a 15% SDS-PAGE and transfer to a PVDF membrane, extracts were probed with the same serous ovarian cancer patient serum as was used to screen the cDNA library, and developed with peroxidase linked anti-human Ig antibody, and lumiglo.

Thus far, only those patients whose tumors express HOXA7 generate serum antibody specifically reactive with HOXA7-expressing recombinant phage (data not shown). Autologous antibody to HOXA7 appears to occur in patients with more histologically differentiated tumors, but not in poorly differentiated tumors or healthy volunteers. While encouraging, these observations need to be extended to more patients and controls.

### Example 8

### Development of ELISA to Detect Human Serum Antibody Specific for HOXB7

The full length HOXB7 gene was cloned in-frame with an N-terminal 6His tag in vector pProExHT_{b} (LTI). E.coli DH5α was transformed with this construct, and HOXB7 expression induced by IPTG induction. Expression of 6His-HOXB7 was confirmed by Western blot (not shown). The recombinant 6His-HOXB7 protein proved soluble, and was affinity purified on NiNTA Sepharose. Bovine papillomavirus (BPV) L2, was also expressed in E.coli and affinity purified using its 6His tag (Roden et al., 1994). BPV L2 was chosen as a control protein to determine background antibody binding because patients are very unlikely to have been exposed to this virus.

Microtiter wells were coated (2 h, 37° C.) with 100 ng of either HOXB7 or Bovine Papillomavirus (BPV) L2 (control) 6 His-tagged antigens purified from E.coli. After washing with PBS, 0.01% Tween 20, non-specific binding was blocked using 200 μl of PBS, 1% milk powder, 0.01% Tween 20 for an hour. This was replaced by 100 μl of ovarian cancer patient (n=39, n=1 (stage II), n=30 (stage III), n—8 (stage IV)) or healthy volunteer (n=29) serum diluted 1:500 in PBS, 1% milk powder for one hour. After five washes, the bound antibody was detected with peroxidase-linked anti human IgG (1:5000, 30 min, RT) and TMB. Absorbance values were significantly elevated in patients compared to controls for HOXB7 responses observed among patients (p<0.0001, Mann-Whitney U test), but not BPV L2 (n.s.). Absorbance values were also significantly different between HOXB7 and BPV L2 reactivity in patients (p<0.0001, Wilcoxon Signed Rank test). Thirteen of 39 patients, but only one of 29 healthy women were found to generate anti-HOXB7 antibodies, where a positive reaction is defined as an optical density value that exceeds the mean optical density value of sera of healthy donors by three standard deviations.

Although sera from patients with early-stage organ-confined disease were not available for analysis, there was no obvious correlation between serologic responses to HOXB7 and disease stage (II to IV). Preliminary studies showed reactivity to the ABC7 protein by the patient serum used to immuno-screen the library, but not by nine other patient sera (data not shown). The generation of anti-HOXB7 antibodies by ovarian cancer patients is consistent with the overexpression of HOXB7 mRNA in ovarian tumor specimens compared to normal ovarian surface epithelium as assessed by semi-quantitative RT-PCR (see Example 7). The growth promoting activity of HOXB7 overexpression in breast carcinoma and melanoma and its ability to up-regulated bFGF expression in these tissues render this observation extremely provocative (Care,A., Silvani, A., Meccia, E., Mattia, G., Peschle, C., and Colombo, M. P., “Transduction of the SkBr3 Breast Carcinoma Cell Line with the HOXB7 Gene Induces bFGF Expression, Increases Cell Proliferation and Reduces Growth Factor Dependence,” Oncogene, 16:3285-3289, 1998; Care et al., 1996).

### Example 9

### Development of ELISA to Detect Human Serum Antibody Specific For HOXA7

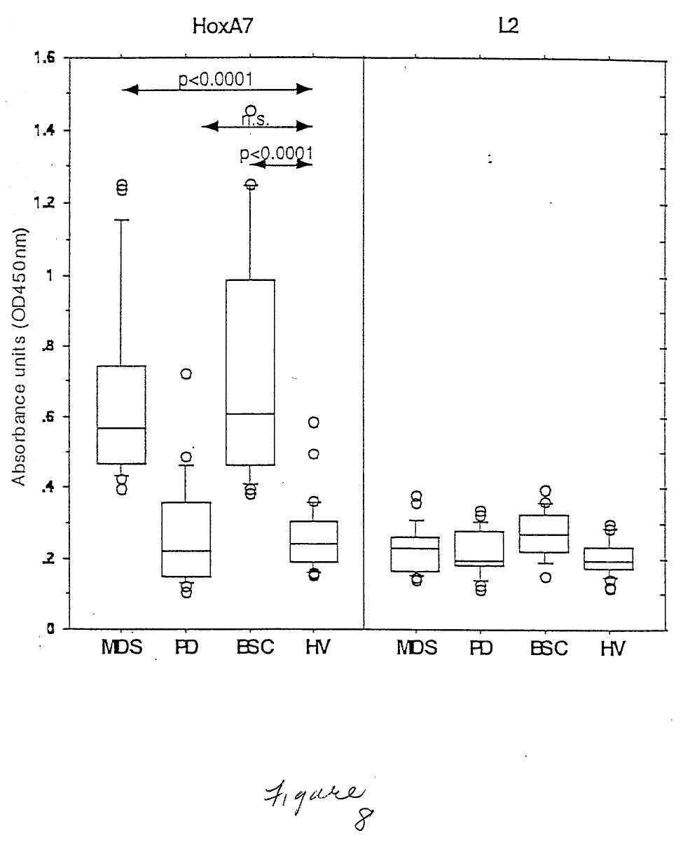

An ELISA was developed for HOXA7 (as for HOXB7, see Example 8). Since immunohistochemical staining and RT-PCR analysis suggested that expression of HOXA7 relates to the differentiation pattern of the tumor, sera of patients were further categorized by the nature of the ovarian tumor. Specifically, sera from patients with moderately differentiated serous carcinoma (MDS, n=24), poorly differentiated ovarian carcinoma (PD, n=24), benign serous ovarian cystadenoma (BSC, n=19) or healthy volunteer (HV, n=30) were tested for reactivity to purified HOXA7 or control protein (BPV L2) (FIG. 8). Microtiter wells were coated (2 h, 37° C.) with 10 ng of either HOXA7 or Bovine Papillomavirus (BPV) L2 (control) 6His-tagged antigens purified from E.coli. Sera from patients with moderately differentiated serous carcinoma (MDS, n=24), poorly differentiated ovarian carcinoma (PD, n=24), benign serous ovarian cystadenoma (BSC, n=19) or healthy volunteer (HV, n=30) were diluted 1:500 and added to each well (100μl) for an hour. After five washes, the bound antibody was detected with peroxidase-linked anti human IgG (1:5000, 30 min, RT) and TMB. Absorbance values were significantly elevated in moderately differentiated serous carcinoma and benign serous cystadenoma patients compared to controls for HOXA7 responses (p<0.0001, Mann-Whitney U test), but not BPV L2 (n.s.). Absorbance values were also significantly different between HOXA7 and BPV L2 reactivity (p<0.005, Wilcoxon Signed Rank test).

This study suggests that a test based upon autologous antibody to HOXA7 would detect moderately differentiated serous ovarian carcinoma, and also benign serous ovarian cystadenoma.

### Example 10

### Effect of HOXB7 Over-Expression on OSE Cell Proliferation

Since HOXB7 expression was markedly higher in carcinomas than in normal OSE and HOXB7 regulates proliferation of several other cell types (25-27), the possibility that HOXB7 over-expression increases proliferation of OSE cells was investigated. IOSE-29 cells were stably transfected with HOXB7 cloned in three different expression vectors. Full-length HOXB7 cDNA was subdloned from pPROEXHTb-HOXB7 into mammalian expression vectors pBK-CMV (Stratagene) and pRESpuro2 (Clontech). In addition, full-length HOXB7 cDNA, cloned in pcDNA3(17), was subdloned into pcDNA3.1 (Invitrogen, Carlsbad, Calif.). Sub-confluent cultures of IOSE-29 cells were transfected with linearized DNA using Lipofectamine PLUS reagent (Life Technologies). Cells transfected with pBK-CMV and pcDNA3.1 constructs were selected with G148 (400 μg/ml) and cells transfected with pIRESpuro2 constructs were selected with puromycin (1 μg/ml). Experiments were performed using lines established from single colonies.

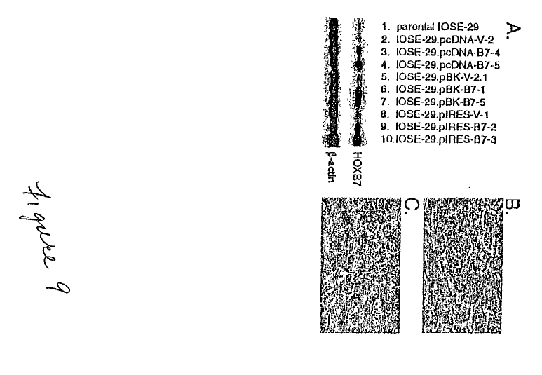

The resulting cell lines expressed HOXB7 at similar levels, 3 to 4-fold the level in parental IOSE-29 cells and in IOSE-29 cells stably transfected with vector DNA clone (see FIG. 9A). RT-PCR analysis detected HOXB7 expression levels in HOXB7-transfected cells (lanes 3, 4, 6, 7, 9, 10) which were markedly higher than in cells transfected with vector DNA alone (lanes 2, 5, 8) and in the parental cell line (lane 1).

Vector-transfected IOSE-29 cells grew in flat monolayers similar to the parental line (FIG. 9B). In contrast, cultures of HOXB7 transfectants exhibited islands of multi-layered overgrowth (FIG. 9C).

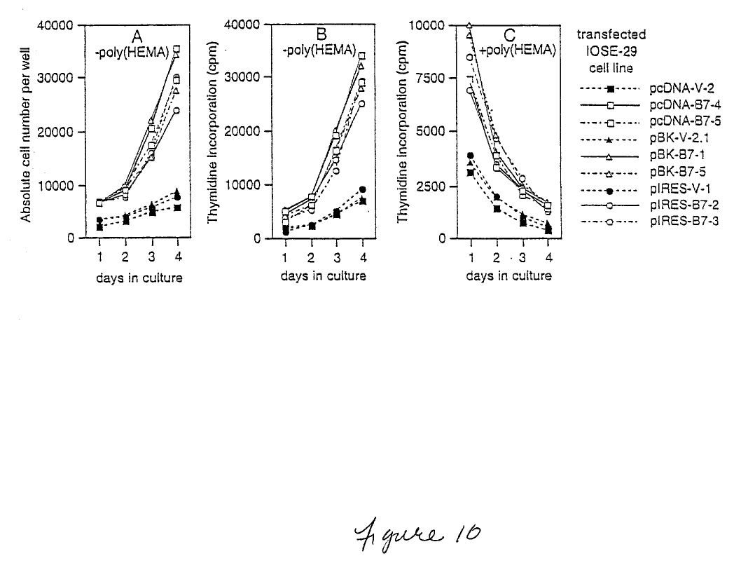

Stably transfected IOSE29 cells were seeded at 2000 cells/200 μl per well in 96-well plates. Total numbers of cells in each uncoated well were counted daily. Thymidine incorporation was measured in cultures pulsed for 3 h with 1 μCi of ³H-methyl-thymidine (60 Ci/mmol) (ICN, Costa Mesa, Calif.) following 1, 2, 3 and 4 days of culture. Shown in FIG. 10 are the mean values of 3-4 independent experiments. Differences in cell numbers and thymidine incorporation levels of HOXB7-transfected cells, as compared with corresponding vector-transfected cells, at each time point were found to be statistically significant (p<0.001). The dramatically enhanced growth of HOXB7 transfectants was evidenced by increases in absolute cell number (FIG. 10A) and in thymidine incorporation (FIG. 10B), which were 3 to 4-fold the levels observed in vector-transfected cells. Cells were also seeded in wells coated with poly 2-hydroxyethylmethacrylate (poly(HEMA)) (Sigma, St. Louis, Mont.) and pulsed for 18 h with ³H-methyl-thymidine. Equivalent numbers of cells of vector- and HOXB7-transfected lines were seeded in wells coated with poly(HEMA) and proliferative activities monitored by measuring thymidine incorporation. Levels of incorporated thymidine in vector-transfected cells progressively declined, reaching almost background levels by Day 4 (FIG. 10C). A similar rate of decline in thymidine incorporation in HOXB7 transfectants was observed (−50% decrease in levels per day), although levels of incorporated thymidine in HOXB7-transfectants were consistently higher than levels in vector-transfectants on any given day (FIG. 10C). An initial increase in numbers of HOXB7-transfected cells during the first 24 h after seeding in poly(HEMA)-coated wells could explain their higher levels of thymidine incorporation, but HOXB7 over-expression in these cells does not appear to permit sustained anchorage-independent growth.

### Example 11

### Effect of HOXB7 Over-Expression on bFGF Production

Growth factor autocrine loops represent a key mechanism regulating tumor cell growth. bFGF has been found by several studies to be expressed in ovarian carcinomas and is widely believed to stimulate their growth (28-30). It was therefore investigated whether over-expression of HOXB7 in OSE cells could up-regulate bFGF production. bFGF levels in culture supernatants collected from equivalent numbers of vector- and HOXB7- transfected IOSE-29 cells and lysates of these cells were assessed by ELISA. Cells were seeded in 25 cm² flasks containing 5 ml medium. Culture supernatants were harvested once cells reached a density of 2×10 cells/cm². Protein lysates were prepared using M-PER reagent (Pierce) at 10⁵ cells/10 μl. bFGF levels were assayed in culture supernatants and cell lysates using the Quantikine human bFGF immunoassay (R&D Systems, Minneapolis, Minn.).

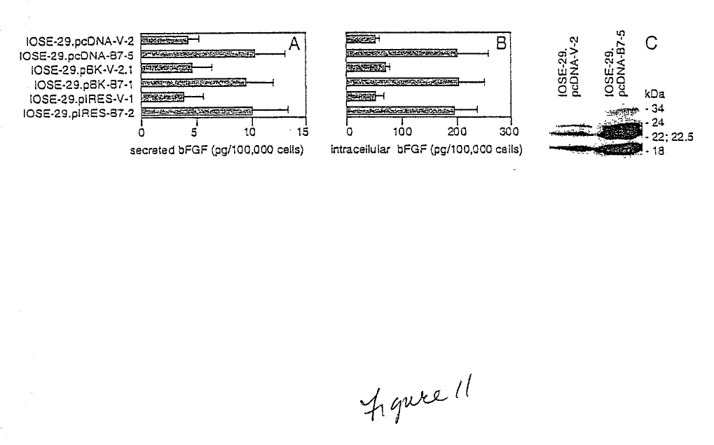

Shown are mean values of 2-3 independent experiments. ELISAs revealed levels of bFGF in culture supernatants of HOXB7-transfected IOSE-29 cells which were approximately three-fold higher than levels in supernatants collected from equivalent numbers of vector-transfected cells (FIG. 11A). The intracellular bFGF content of HOXB7 transfectants was also approximately three-fold higher than that of vector-transfected cells (FIG. 11B). Surprisingly, when the amount of secreted and intracellular bFGF were compared on a per cell basis, the vast majority (˜95%) of total bFGF produced by a cell was intracellular (compare FIG. 11A with FIG. 11B).

Human bFGF is produced naturally in several isoforms (18, 22, 22.5, 24 and 34 kD) that originate from alternative translation initiation sites within a single MRNA species(31, 32). Western blot analysis of lysates (10 μg per lane) of vector-transfected (IOSE-29.pcDNA-V-2) and HOXB7-transfected (IOSE-29.pcDNA-B7-5) IOSE-29 cells were performed. Monoclonal anti-human bFGF (Sigma, clone FB-8) was used for Western blots. Molecular weights of bFGF isoforms are indicated on FIG. 11C. Western blot analysis of cell lysates revealed increased levels of each of these bFGF isoforms in HOXB7-transfected cells, indicating that overexpression of HOXB7 up-regulates total bFGF production in OSE cells.

1. A substantially pure nucleic acid which is homologous with a EcoRI/XhoI fragment isolated from bacteriophage λ clone 44B.1 deposited under ATCC accession No. [N], wherein homologous sequences have a sequence alignment score of greater than 200 as calculated by BLASTN 2.1.2.

2. A substantially pure nucleic acid which hybridizes to a EcoRI/XhoI fragment isolated from bacteriophage λ clone 44B.1 deposited under ATCC accession No. [N] under stringent hybridization conditions, wherein stringent hybridization conditions comprise 0.5 MNaHPO₄/1 mM EDTA/7%(w/v) SDS at 55° C.

3. The nucleic acid of claim 2, wherein hybridization conditions include hybridization at 60° C.

4. The nucleic acid of claim 2, wherein hybridization conditions include hybridization at 65° C.

5. A substantially pure nucleic acid which encodes the amino acid sequence encoded by the EcoRI/XhoI fragment isolated from bacteriophage λ clone 44B.1 deposited under ATCC Accession No. [N].

6. The nucleic acid of claim 1 or 2, wherein the nucleic acid is RNA.

7. The nucleic acid of claim 1 or 2, wherein the nucleic acid is produced by recombinant methods.

8. The nucleic acid of claim 1 or 2, wherein the nucleic acid contains at least 15 nucleotides.

9. A pair of nucleic acid primers comprising at least 10 contiguous nucleotides selected from or complementary to portion of a EcoRI/XhoI fragment isolated from bacteriophage λ clone 44B.1 deposited under ATCC accession No. [N], wherein nucleic acid amplification using the pair of nucleic acid primers will produce an amplified nucleic acid comprising at least 18 contiguous nucleotides of the EcoRI/XhoI fragment isolated from bacteriophage λ clone 44B.1 deposited under ATCC accession No. [N].

10. A replicon comprising a sequence of at least 18 contiguous nucleotides selected from the sequence of a EcoRllXhol fragment isolated from bacteriophage λ clone 44B.1 deposited under ATCC accession No. [N] or its complement under control of a promoter.

11. A recombinant cell containing the replicon of claim 10.

12. A substantially pure polypeptide comprising an amino acid sequence encoded by a EcoRI/XhoI fragment isolated from bacteriophage λ clone 44B.1 deposited under ATCC accession No. LN, wherein said polypeptide comprises at least one epitope.

13. The polypeptide of claim 12, wherein the polypeptide contains at least 9 amino acids.

14. The polypeptide of claim 12, wherein the polypeptide consists essentially of an amino sequence encoded by said EcoRI/XhoI fragment.

15. An antibody which specifically binds to a mammalian protein comprising an amino acid sequence encoded by a EcoRI/XhoI fragment isolated from bacteriophage λ clone 44B.1 deposited under ATCC accession No. [N].

16. The antibody according to claim 15, wherein the antibody is in an isolated polyclonal antiserum, a preparation of purified polyclonal antibodies, or a preparation containing one or more monoclonal antibodies.

17. A method for selecting variant nucleic acid sequences comprising (a) screening mammalian DNA or RNA with a nucleic acid probe comprising the nucleic acid of claim 1 or claim 2, (b) sequencing the DNA or RNA obtained in said screening, and (c) selecting DNA or RNA having sequences that differ from the nucleic acid sequence in a EcoRI/XhoI fragment of bacteriophage λ clone 44B.1 deposited under ATCC accession No. [N] by at least one nucleotide.

18. A method of screening for cancer in an individual comprising determining whether cells in the individual are expressing a gene product encoded by a EcoRI/XhoI fragment isolated from bacteriophage λ clone 44B.1 deposited under ATCC accession No. [N], expression of this product being correlated with an increased likelihood of cancer in the individual.

19. A nucleic acid encoding HOXB7 having an amino acid sequence corresponding to the amino acid sequence encoded by the sequence shown as SEQ ID NO: 1.

20. A protein encoded by the nucleic acid of claim 19.

21. A method of screening for cancer in an individual comprising determining whether cells in the individual are expressing a product encoded by the nucleic acid of claim 19, expression of this product being correlated with an increased likelihood of cancer in the individual.

22. A method of screening for cancer other than breast cancer or melanoma in an individual comprising determining whether cells in the individual are expressing a gene product of HOXB7 gene, expression of this gene product being correlated with an increased likelihood of cancer in the individual.

23. A method of screening for cancer other than renal cell carcinoma or colon cancer in an individual comprising determining whether cells in the individual are expressing a gene product of HOXA7 gene, expression of this gene product being correlated with an increased likelihood of cancer in the individual.

24. A method of screening for benign serous cystadenoma in an individual comprising determining whether cells in the individual are expressing a gene product of HOXA7, expression of this gene product being correlated with an increased likelihood of benign serous cystadenoma in the individual.

25. A method of screening for ovarian neoplasm in an individual comprising determining whether cells in the individual are expressing a gene product of HOXA7, expression of this gene product being correlated with an increased likelihood of ovarian neoplasm in the individual.

26. The method of claim 25, wherein the ovarian neoplasm is ovarian cancer.

27. The method of claim 25, wherein the ovarian neoplasm is benign serous cystadenoma.

28. A method of screening for cancer in an individual comprising determining whether cells in the individual are expressing a product consisting of ATP-dependent iron transporter ABC-7, expression of this product being correlated with an increased likelihood of cancer in the individual.

29. A method of screening for cancer in an individual comprising determining whether cells in the individual are expressing a product consisting of ADP-ribosylation factor 1 (Arf-1), expression of this product being correlated with an increased likelihood of cancer in the individual.

30. A method of screening for cancer in an individual comprising determining whether cells in the individual are simultaneously expressing two or more gene products selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage clone 44B.1 deposited under ATCC accession No. [N], expression of a plurality of these gene products being correlated with an increased likelihood of cancer in the individual.

31. The method according to any one of claim 18 or 21-30, wherein said method comprises
providing a histologic section of tissue from the individual;
contacting said histologic section with antibody which specifically binds said product; and
determining said antibody specifically binds to the histologic section, whereby specific binding of said antibody to the histologic section correlates with increased likelihood of cancer in the individual.

32. The method according to any one of claim 18 or 21-30, wherein said method comprises
providing a sample of tissue from the individual; and
determining, in said sample, level of expression of a gene product having the sequence of said product, whereby expression of said product in the sample correlates with increased likelihood of cancer in the individual.

33. The method of claim 32, wherein the gene product is mRNA.

34. The method of claim 33, wherein the mRNA is extracted from said sample and quantitated.

35. The method of claim 33, wherein the level of mRNA is determined by in situ hybridization to a section of the tissue sample.

36. The method of claim 33, wherein the mRNA is quantitated by reverse transcriptase-polymerase chain reaction.

37. The method of claim 32 wherein the tissue is a carcinoma.

38. The method of claim 37, wherein the carcinoma is ovarian cancer.

39. A kit for screening human samples according to the method of claim 31, comprising in one or more containers;
an antibody which specifically binds to one or more epitopes found on said product; and a reagent means for detecting the antibody.

40. A kit for screening human samples according to the method of claim 32, comprising in one or more containers:
a nucleotide probe which hybridizes to MRNA encoding said product; and reagent means for detecting the nucleotide probe.

41. A method of screening for cancer in an individual comprising obtaining a sample of bodily fluid from the individual and determining whether or not the sample contains antibodies specific for one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage λ clone 44B.1 deposited under ATCC accession No. [N], the presence of antibodies to any one of these proteins being correlated with an increased likelihood of cancer in the individual.

42. A method of screening for cancer in an individual comprising obtaining a sample of bodily fluid from the individual and determining whether or not the sample contains one or more proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage λ clone 44B. 1 deposited under ATCC accession No. [N], the presence of any one of these proteins in the bodily fluid being correlated with an increased likelihood of cancer in the individual.

43. A method of cancer therapy comprising immunizing an individual with an immunogenic composition to elicit an immune response to one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage X clone 44B.1 deposited under ATCC accession No. [N].

44. The method of claim 43, wherein the immune response is a cellular immune response.

45. The method of claim 43, wherein the immunogenic composition comprises at least one epitope of one or more of the proteins selected from the group consisting of homeobox protein HOXA7, homeobox protein HOXB7, ADP-ribosylation factor 1 (Arf-1), ATP-dependent iron transporter ABC-7, and the protein encoded by a EcoRI/XhoI fragment of bacteriophage λ clone 44B.1 deposited under ATCC accession No. [N].


SEQUENCE LISTING (8 sequences)

SEQ ID NO: 1 - DNA, 753 bp, Homo sapiens
  CDS (1)...(654)
atgagttcattgtattatgcgaatgctttattttctaaatatccagcctcaagttcggttttcgctaccggagccttcccagaacaaacttcttgtgcgtttgcttccaacccccagcgcccgggctatggagcgggttcgggcgcttccttcgccgcctcgatgcagggcttgtaccccggcggggggggcatggcgggccagagcgcggccggcgtctacgcggccggctatgggctcgagccgagttccttcaacatgcactgcgcgccctttgagcagaacctctccggggtgtgtcccggcgactccgccaaggcggcgggcgccaaggagcagagggactcggacttggcggccgagagtaacttccggatctacccctggatgcgaagctcaggaactgaccgcaaacgaggccgccagacctacacccgctaccagaccctggagctggagaaagaatttcactacaatcgctacctgacgcggcggcggcgcatcgagatcgcgcacacgctctgcctcacggaaagacagatcaagatttggtttcagaaccggcgcatgaagtggaaaaaggagaacaagaccgcgggcccggggaccaccggccaagacagggctgaagcagaggaggaagaggaagagtgagggatggagaaagggcagaggaagagacatgagaaagggagaggaagagaagcccagctctgggaactgaatcaggaaactcaaatcgaatagggaagt

SEQ ID NO: 2 - DNA, 20 bp, Artificial Sequence
  Primers
gagctggagaaggagttcca

SEQ ID NO: 3 - DNA, 20 bp, Artificial Sequence
  Primers
ctttcttccacttcatacga

SEQ ID NO: 4 - DNA, 19 bp, Artificial Sequence
  Primers
agagtaacttccggatcta

SEQ ID NO: 5 - DNA, 19 bp, Artificial Sequence
  Primers
tctgcttcagccctgtctt

SEQ ID NO: 6 - DNA, 18 bp, Artificial Sequence
  Primers
atgatatcgccgcgctcg

SEQ ID NO: 7 - DNA, 19 bp, Artificial Sequence
  Primers
cgctcggtgaggatcttca

SEQ ID NO: 8 - protein, 14 aa, Homo sapiens
DKADEGVLHGPAEA